(19) United States
(12) Patent Application Publication
(10) Pub. No.: US 2025/0143680 A1
(43) Pub. Date: May 8, 2025

(54) SURGICAL INSTRUMENT ASSEMBLY

(71) Applicant: FlexDex, Inc., Brighton, MI (US)

(72) Inventors: Zachary R. Zimmerman, Brighton, MI (US); Matthew P. Weber, Brighton, MI (US); James Duncan Geiger, Ottawa Hills, OH (US); Rogelio Insignares, Howell, MI (US); Christopher Paul Huang Shu, Ann Arbor, MI (US); Brian Jeffery, Brownstown, MI (US)

(21) Appl. No.: 18/936,886

(22) Filed: Nov. 4, 2024

Related U.S. Application Data
(60) Provisional application No. 63/547,037, filed on Nov. 2, 2023.

(51) Int. Cl. A61B 17/00; A61B 90/00

(52) U.S. Cl. CPC A61B 17/00234; A61B 90/08; A61B 2017/00367; A61B 2017/00955; A61B 2090/0803; A61B 2090/0807

(57) ABSTRACT

A surgical instrument assembly is provided that can be equipped in a handheld surgical tool or instrument or other medical device, as well as potentially in non-medical devices, among other possible applications. In various implementations, the surgical instrument assembly has combinations of a frame body, an input body, an indexing body, a tensioning body, and a cover body. The indexing body is moveable to a multitude of indexed positions. The indexed positions constitute a use count of the surgical instrument assembly, and a final indexed position is a final use count of the surgical instrument assembly. The tensioning body is moveable to impart a first tension and a second tension to one or more transmission cables. The cover body is moveable to render a flush port inaccessible and accessible.

### CROSS-REFERENCE TO RELATED APPLICATION

This application claims the benefit of U.S. Provisional Patent Application No. 63/547,037, with a filing date of Nov. 2, 2023, the contents of which are hereby incorporated by reference in their entirety.

### TECHNICAL FIELD

The present disclosure relates generally to surgical instrument assemblies, for example handheld surgical instrument assemblies that can be employed for use in minimally invasive surgical (MIS) procedures.

### BACKGROUND

Surgical tools are often designed and constructed with various components to have certain kinematic architectures at the handle and frame, and to ultimately furnish certain functionalities and performances at an end effector. Drawbacks can arise among the architectures depending on how the handle and frame and components are arranged and configured with respect to one another.

Reusable surgical medical devices often require data storage of the number of times the device has been used during surgical procedure for device lifecycle management. This data is stored externally for standard surgical instruments. This may include a pen and paper tracking method, a spreadsheet, a data reading device with accompanying software, etc. But this method is not always reliable as it may be skipped or become untethered from the device.

One well-established usage counting means involves scanning machine-readable codes, e.g., QR codes, to identify a device, increment a count in the external data storage, and warn users when the device is approaching end of life.

Another usage counting means involves a built-in device counter that increments as the user activates an input on the device. This input can be a user input that is part of the primary usage mechanism of the device, or it can be a separate mechanism individually activated by the user. For example, a medical inhaler for aerosolized medicine delivery may have a built-in counter that increments when the user performs an action to deliver the medication, or it can have a separate feature that can be engaged independently to increment a count.

Reusable medical devices often have a limited amount of use before they must be reprocessed or made obsolete, either to avoid degradation of device performance or of material integrity. The ability to warn the user that the device is approaching the end of usable life can be critical in certain circumstances to ensure patient safety. Additionally, a use error or a misuse scenario may lead to user continuing device usage past the end of life of the device. In these scenarios, hazards to patient and operator safety could arise. To avoid these risks, medical device manufacturers may choose to integrate obsolescence features to their device. One such means to perform obsolescence may involve preventing a critical device mechanism from activating once certain criteria have been reached. This can be accomplished through electronic disengagement, mechanical movement restriction, or planned failure of a component. For example, a disposable camera may prevent film advancement once the remaining film count has incremented to zero by deactivating the advancing mechanisms software, physically breaking the advancement gear pawl, or engage a lock-out pawl that prevents advancement gear rotation. Another example may be a single use sterile glucose test strip that interfaces with glucose testing machines. The engagement feature may be warped or broken as part of the insertion or usage process, preventing non-sterile reuse of the device.

Medical devices containing long narrow lumens often pose a cleanability challenge. Many laparoscopic devices which incorporate such elongate shaft members also include a method to boost cleanability within the long narrow lumens such as a flush port. This flush port can be exposed to patient blood and tissue during surgical application, which in turn adds bioburden and introduces new cleanability and drainage challenges. Device manufacturers may incorporate one-way valves, physical covers, or port seals to mitigate the contamination risk. But each one of these solutions may also decrease the effectiveness of the flush port.

Cable-driven medical instruments can pose additional reprocessing difficulties. For instance, due to thermal expansion between materials of different thermal properties, a cable-driven device is subjected to additional stress in high temperature sterilization processes. Therefore, for safe reprocessing, reusable instruments with cable-driven mechanisms may need a tension-relief feature to extend the device usage life.

Several of these mechanisms suitable for a reusable medical device have been described in this section, but user compliance and usability decrease as the amount of required user actions and reprocessing complexity increases. To ensure patient safety, there is a need for a streamlined solution that incorporates medical device life cycle management, obsolescence, flush port protection, and device tension relief.

### SUMMARY

In an embodiment, a surgical instrument assembly may include a frame body, an input body, and an indexing body. The input body can be moved with respect to the frame body. The input body has a first position with respect to the frame body, and has a second position with respect to the frame body. The indexing body is prompted to move with respect to the frame body by way of the input body. The indexing body can be moved to a multitude of indexed positions with respect to the frame body. Upon attainment by the indexing body of a final indexed position of the multitude of indexed positions, movement of the input body with respect to the frame body is fully constrained.

In another embodiment, a surgical instrument assembly may include a frame body, an input body, and a tensioning body. The input body can be moved with respect to the frame body. The input body has a first position with respect to the frame body, and has a second position with respect to the frame body. The tensioning body is prompted to move with respect to the frame body by way of the input body. The surgical instrument assembly has a use state and a non-use state. The use state can be established when the input body is in the first position, and the non-use state can be established when the input body is in the second position. When the surgical instrument assembly is in the use state, a first tension is provided to one or more transmission cables by way of the tensioning body. Movement of the input body from the first position and to the second position provides a second tension to the transmission cable(s) by way of the tensioning body. The second tension is less than the first tension.

In another embodiment, a surgical instrument assembly may include a frame body, an input body, and an indexing body. The input body can be moved with respect to the frame body. The indexing body is prompted to move with respect to the frame body by way of the input body. The indexing body can be moved to a multitude of indexed positions with respect to the frame body. Each of the multitude of indexed positions constitutes a use count of the surgical instrument assembly. Advancement of the indexing body to each of the multitude of indexed positions serves to increment the use count of the surgical instrument assembly.

In another embodiment, a method of counting uses of a surgical instrument assembly may include numerous steps. One step involves moving an input body with respect to a frame body from a first position and to a second position. Movement of the input body from the first position and to the second position serves to prompt movement of an indexing body with respect to the frame body to an indexed position. The indexed position is of a multitude of indexed positions. When the input body is in the first position, a use state of the surgical instrument assembly is established. When the input body is in the second position, a reprocessing-ready state of the surgical instrument is established. Another step of the method involves incrementing a use count of the surgical instrument assembly upon advancement of the indexing body to each of the multitude of indexed positions and upon establishment of each reprocessing-ready states of the surgical instrument assembly.

In another embodiment, a method of de-tensioning a surgical instrument assembly may include numerous steps. One step involves moving an input body to a first position with respect to a frame body. A use state of the surgical instrument assembly is established when the input body is in the first position and a first tension is provided to one or more transmission cables by way of a tensioning body. Another step of the method involves moving the input body to a second position with respect to the frame body. A non-use state of the surgical instrument assembly is established when the input body is in the second position and a second tension is provided to the transmission cable(s) by way of the tensioning body. The second tension is less than the first tension.

In another embodiment, a handheld surgical instrument assembly may include a handle assembly, a frame body, a shaft body, a flush port, an end effector assembly, and one or more transmission cables. The flush port is in fluid communication with the shaft body. The transmission cable(s) extends from the handle assembly to the end effector assembly. The transmission cable(s) transmits actions to the end effector assembly. An input body can be moved with respect to the frame body. An indexing body can be moved with respect to the frame body. Movement of the indexing body serves to effect a use count of the handheld surgical instrument assembly. A tensioning body can be moved with respect to the frame body. Movement of the tensioning body serves to effect a tension of the transmission cable(s). A cover body can be moved with respect to the frame body. Movement of the cover body serves to effect accessibility and inaccessibility of the flush port. A single user input action to the input body and movement of the input body with respect to the frame body concurrently moves the indexing body with respect to the frame body, moves the tensioning body with respect to the frame body, and moves the cover body with respect to the frame body.

In another embodiment, a surgical instrument assembly may include a frame body, an input body, an indexing body, and an obsolescence body. The input body can be moved with respect to the frame body. The indexing body is prompted to move with respect to the frame body by way of the input body. The obsolescence body can be moved with respect to the frame body. Upon movement of the obsolescence body, movement of the input body with respect to the frame body is fully constrained. Movement of the obsolescence body and full constraint of the input body with respect to the frame body is prompted by way of attainment of a final indexed position of the indexing body, or is prompted by way of an event trigger.

Further scope of applicability of the present disclosure will become apparent from the detailed description given hereinafter. But it should be understood that the detailed description and specific examples, while indicating exemplary embodiments of the disclosure, are given by way of illustration only, since various changes and modifications within the spirit and scope of the disclosure will become apparent to those skilled in the art from this detailed description

### BRIEF DESCRIPTION OF THE DRAWINGS

The present disclosure will become more fully understood from the detailed description given below and the accompanying drawings, which are given by way of illustration only, and do not limit the present disclosure, and wherein:

FIG. 1 is a block diagram of an embodiment of a surgical instrument assembly;

FIG. 2 is a block diagram of another embodiment of the surgical instrument assembly;

FIG. 3 is a block diagram of another embodiment of the surgical instrument assembly;

FIG. 4a is a diagrammatic representation of an embodiment of use and non-use states of the surgical instrument assembly;

FIG. 4b is a diagrammatic representation of an embodiment of a use state of the surgical instrument assembly;

FIG. 4c is a diagrammatic representation of an embodiment of a non-use state of the surgical instrument assembly;

FIG. 5 is a diagrammatic representation of an embodiment of a tensioning functionality of the surgical instrument assembly;

FIG. 6 is a diagrammatic representation of an embodiment of angular positions of an indexing body, a tensioning body, and a flush port cover body of the surgical instrument assembly;

FIG. 7 is a diagrammatic representation of an embodiment of an advancement of use and non-use states of an indexing body and a ratchet wheel body of the surgical instrument assembly;

FIG. 8 is a side view of an embodiment of a handheld surgical instrument;

FIG. 9 is an enlarged view of the handheld surgical instrument in a use state thereof;

FIG. 10 is an enlarged view of the handheld surgical instrument in a non-use state thereof;

FIG. 11 is a sectional view of certain components of the handheld surgical instrument;

FIG. 12 is an enlarged and partially segmented view of components of the handheld surgical instrument in the non-use state;

FIG. 13 is a sectional view of certain components of the handheld surgical instrument;

FIG. 14 is a sectional view of certain components of the handheld surgical instrument;

FIG. 15a is a sectional view showing an embodiment of a tensioning body and other components of the handheld surgical instrument in the use state;

FIG. 15b is a sectional view of the tensioning body and other components of the handheld surgical instrument in the non-use state;

FIG. 15c is an isolated view of the tensioning body and a transmission cable interacting therewith, demonstrating the use state and non-use state of the handheld surgical instrument;

FIG. 16a is a sectional view of an embodiment of an input body or counter ring body or counter knob body of the handheld surgical instrument;

FIG. 16b is a diagrammatic representation of the input body or counter ring body or counter knob body of the handheld surgical instrument;

FIG. 17a is an isolated view of an embodiment of an indexing body or ratchet wheel body of the handheld surgical instrument;

FIG. 17b is a diagrammatic representation of the indexing body or ratchet wheel body of the handheld surgical instrument;

FIG. 18a is a sectional view of an embodiment of a frame body of the handheld surgical instrument; and

FIG. 18b is a diagrammatic representation of the frame body of the handheld surgical instrument.

### DETAILED DESCRIPTION

Multiple embodiments of surgical instrument assemblies and handheld surgical instrument assemblies and medical devices are depicted in the figures and detailed in this description. Definitions of certain technical terms used herein are presented prior to particular figure references in this description:

Body-A body is a discrete continuous component that can be used as structural components to form an assembly or sub-assembly. The displacement/motion state of a body can be completely defined with respect to a reference ground by six degrees of freedom (DoF). A body may be rigid or compliant. One or more discrete bodies may be connected via joints to form an assembly. These bodies, together as an assembly, may still be termed as a body. In certain scenarios, a body may be produced out of many discrete rigid bodies joined by rigid joints. The resulting body, alternatively, may also be a single/monolithic structure. In certain scenarios, a body may be compliant but still discrete and continuous.

Mechanism/Joint/Connector—In general, there may be a certain equivalence between the terms “mechanism” and “joint.” A “joint” may be alternatively referred to as a “connector” or a “constraint.” All of these can be viewed as allowing certain rigid body motion(s) along certain degree(s) of freedom between two bodies and constraining the remaining motions. A mechanism generally comprises multiple joints and bodies. Typically, a joint may be of simpler construction, while a mechanism may be more complex as it can comprise multiple joints. Generally, a joint refers to a mechanical connection that allows motions as opposed to a fixed joint (e.g., welded, bolted, screwed, or glued jointly). In the latter case, fixed joint, two bodies are fused with each other and are considered one and the same in the kinematic sense (because there is no relative motion allowed or there are no relative degrees of freedom between the two bodies). The term “fixed joint” may be used herein to refer to this kind of joint between two bodies. When reference to the term “joint” is made, it means a connection that allows at least some motions or degrees of freedom, e.g., a pin joint, a pivot joint, a universal joint, a ball and socket joint, etc.

Degree of Freedom (DoF)—As noted, a joint or mechanism allows certain rigid body motions between two bodies and constrains the remaining motions. “Degrees of freedom” is a technical term to capture or convey these allowed “motions.” In total, there are six independent motions and therefore six degrees of freedom possible between two rigid bodies when there is no joint between them: three translational DoFs and three rotational DoFs. A joint will allow anywhere between zero and six DoFs between the two bodies. For the case when the joint allows zero DoFs, this effectively becomes a “fixed joint,” as described above, where the two bodies are rigidly fused or connected to each other. In this case, from a kinematic sense, the two bodies are one and the same. For the case when the joint allows six DoFs, this effectively means that the joint does not constrain any motions between the two bodies. In other words, the motions of the two bodies are entirely independent of each other.

Degree of Constraint (DoC)—“Degree of constraint” refers to directions along which relative motion is constrained between two bodies. Since relative motion is constrained, these are directions along which motion and loads (i.e., forces or moments) can be transmitted from one body to the other body. Since the joint does not allow relative motion between the two bodies in the DoC direction, if one body moves in the DoC direction, it drives along with it the other body along that direction. In other words, motions are transmitted from one rigid body to another in the DoC directions. Consequently, loads are also transmitted from one rigid body to another in the DoC directions, which are sometimes also referred to as the load bearing directions or simply bearing directions. The term “retention” may also be used in the context of a DoC direction. For example, one body may be constrained or equivalently retained with respect to a second body along a certain DoC. This means that relative motion is not allowed between the two bodies in the DoC direction, or equivalently the direction of constraint, or equivalently the direction of retention. Retention of all six DoFs means the same thing as having six DoCs between two bodies.

Local Ground—In the context of an assembly of bodies connected by joints (e.g., a multi-body system, a mechanism), one or more bodies may be referred to as the “reference” or “ground” or “local ground.” The body referred to as the local ground is not necessarily an absolute ground (i.e., attached or bolted to the actual ground). Rather, the body that is selected as a local ground simply serves as a mechanical reference with respect to which the motions of all other bodies are described or investigated.

Axial Design—A configuration or arrangement where the primary components or elements are aligned along a common central axis. In this design, the central axis acts as a reference line around which the components are organized or positioned. This axial alignment enables efficient transfer of forces or signals along the axis, and it is commonly used in various applications such as mechanical systems, electrical connectors, or structural designs where linear alignment and axial symmetry are desired.

Radial Design—A configuration or arrangement where the primary components or elements are positioned around a central point or axis, radiating outward in a circular or spoke-like pattern. In this design, the central point serves as the focal point from which the components span outward in a symmetrical manner. Radial designs are commonly used in various applications such as mechanical systems, electronic circuit boards, or architectural layouts where balanced distribution, equal access, or efficient energy transfer from a central source are desired.

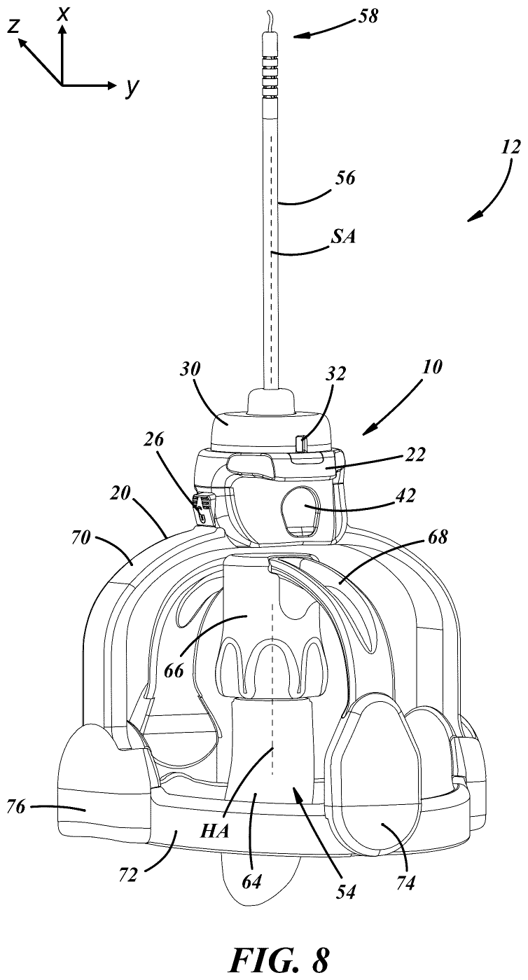

Axis and Direction—Axis refers to a specific line in space. A body may rotate with respect to (w.r.t.) another body about a certain axis. Alternatively, a body may translate w.r.t. another body in a certain direction. A direction is not defined by a particular axis and is instead commonly defined by multiple parallel axes. Thus, x-axis is a specific axis defined in space, while X direction refers to the direction of the x-axis or any other axis that is parallel to the x-axis. Different but parallel axes can have the same X direction. Direction only has an orientation and not a location in space. In at least some embodiments, and with particular reference to FIG. 8, a coordinate system is presented with the x-axis coinciding with an axis of a shaft body or tool shaft of the handheld surgical instrument, the y-axis oriented relative thereto, and the z-axis oriented relative thereto. Here, the x-axis can also be referred to as a roll axis when the particular device rolls about the axis, the y-axis is established by a pitch axis, and the z-axis is established by a yaw axis.

Device Frame—When provided in an embodiment, the device frame refers to a structural body or subassembly, which may have relative motion amongst components, that may be part of a tool apparatus or surgical tool or handheld surgical instrument. In certain tool apparatuses, it may be connected to a handle assembly or handle body and/or an elongated device shaft or shaft body. Terms namely “device frame” and “frame” and “frame body” may be used interchangeably throughout the patent.

Device Shaft—When provided in an embodiment, a device shaft is generally a rigid extension of the frame, at its proximal end, which is a slender and elongated member, commonly a cylinder. An output body, such as an end-effector assembly or end effector body, may be constrained to the distal end of the device shaft. The tool shaft may simply be referred to as a shaft or shaft body. The axis of the tool shaft may be referred to as Device Shaft Axis throughout the description.

Counting Mechanism—Mechanism designed to keep track of the number of times a device has been used. This single counting mechanism may be capable of displaying various data. The use state of the device may be displayed for field use applications, additionally the cycle count may be displayed for reprocessing applications.

Thermal Counting Mechanism—A counting mechanism that utilizes changes in temperature to track and record counts. It operates on the principle that certain materials (such as nitinol or bimetallic strips) exhibit temperature-dependent characteristics, such as expansion or contraction, which interacts with an additional body resulting in a count reading.

Rotary Gear Display—Type of counting mechanism that utilizes rotating gears to visually indicate and track counts. The mechanism consists of interconnected gears with teeth or markings that increment or decrement with each rotation. As the gears rotate, the count is visually displayed by observing the position or alignment of the teeth or markings, providing a clear and intuitive representation of the count value.

Non-Back Drivable—A mechanical or electronic system that prevents reverse movement or unintended changes in its count. It is designed to ensure that once a count increment or decrement occurs, it cannot be easily reversed or manipulated by external forces or back-driving.

Transmission Member Tensioning Mechanism—Refers to a system or mechanism designed to adjust and maintain the tension of cables used within the device. It ensures that the cables are appropriately tightened to achieve optimal performance and functionality.

End of Life (E.O.L.)—The stage in its lifecycle where it is no longer supported, maintained, or used in clinical practice. It signifies the cessation of production, availability of spare parts, and official technical support from the manufacturer. The end of life phase can be prompted by factors such as technological advancements, regulatory changes, or the device reaching its expected operational lifespan.

Flush Port Exposure—In medical devices, this refers to the vulnerability that arises when a communication port on the device is directly accessible from the outside without proper security measures. This can lead to unauthorized access, tampering with patient data, and the injection of malicious commands. To mitigate these risks, robust security practices such as encryption, access controls, and regular security assessments should be implemented, along with network segmentation and firewalls to isolate the device from potentially malicious network traffic.

Use Condition—The Use Condition of the device is also defined by State A. Within this state the device is ready to be used in the field such that, per an embodiment, the device is tensioned, Counter displays the Use Status, and the Flush port is concealed. The device being tensioned means that forces and displacement from the Handle Body is able to be transferred to the End Effector Body. The Use status is a display icon which signals to the user that the device is ready for use. While concealing the flush port helps prevent excess bioburden from entering the flush port while in use.

Reprocessing/Storage Condition—The Reprocessing or Storage Condition of the device is also defined by State A′. Within this state, per an embodiment, the device is de-tensioned, Counter displays the remaining uses/indexed count, and the flush port is exposed. When the device is in the de-tensioned state the forces and displacements of the Transmission members are unable to effectively transfer input from the Handle Body to the End Effector Body, therefore the device is not fully operational. When in this State A′ the counter display may display an indexed value which can distinguish the life remaining in the device. Additionally, by being in State A′ the device can be reprocessed since the Flush Port is now exposed and able to be accessed within a cleaning procedure.

Obsolescence—The process where the device becomes outdated, no longer supported, or technologically obsolete. It occurs when newer and more advanced technologies, treatments, or regulatory standards surpass the capabilities of the device, rendering it less effective or unreliable. This may present risks to patient safety as the device may lack necessary updates, compatibility with other systems, or the ability to address emerging medical challenges. Proper management includes monitoring the device lifecycle, planning for upgrades or replacements, and ensuring regulatory compliance and patient safety throughout the transition to newer technologies.

Reprocessing—Refers to the sterile processing of a reusable medical device to eliminate contaminants and ensure compliance with applicable cleanliness, functionality, and sterility standards. Reprocessing may involve manual or automated procedures and employs techniques such as mechanical cleaning, lubrication, chemical disinfection, and thermal sterilization.

Embodiments described herein have several features, no single one of these features is solely responsible for their desirable attributes. Without limiting the scope of the disclosure, some of the advantageous features will now be discussed briefly.

With reference now to the figures, embodiments of a surgical instrument assembly 10, also called a handheld surgical instrument assembly per certain applications and implementations, are described and depicted herein. The surgical instrument assembly 10 can be equipped in a handheld surgical tool or instrument 12 (FIG. 8) or other medical device, as well as in non-medical devices, among other possible applications. When in the handheld surgical instrument 12, the surgical instrument assembly 10 is referred to as the handheld surgical instrument assembly and can be employed for use in minimally invasive surgical (MIS) procedures. In at least some embodiments presented below, and in the application of the handheld surgical instrument 12 for use in an MIS procedure, the handheld surgical instrument assembly can serve to minimize or altogether rid thermal expansion stress to accompanying transmission cables amid reprocessing procedures such as in an autoclave machine or steam sterilization via a cable tension relief function, track usage and end-of-life progress of the handheld surgical instrument assembly amid its employment in medical procedures and settings, prevent contaminant infiltration through an accompanying flush port, and/or prevent user-access to the flush port. Indeed, per an embodiment in which all of these functions are combined together and provided in the same handheld surgical instrument, a single input action (e.g., single user input action) to a single input body can concurrently initiate and incite all of the functions, thereby in this instance minimizing user effort and mental load. Further, per an embodiment, the handheld surgical instrument can have an obsolescence function that precludes unwanted additional usage of the instrument that would surpass the intended useful life of the instrument; this function can be incorporated with the usage tracking function, per an embodiment of the surgical instrument assembly 10. A sterility protection and device protection can be better ensured during reprocessing procedures, per an embodiment, with one or more or all of these functions provided in the surgical instrument assembly 10. User and patient safety are therefore enhanced in a medical setting. Overall, a more effective and efficient handheld surgical instrument is provided.

In various embodiments, the surgical instrument assembly 10 can have a use count mechanism or assembly, an obsolescence mechanism or assembly, a tensioner or tensioning mechanism or assembly, and/or a flush port inaccessibility and accessibility mechanism or assembly, all described below in greater detail. The use count mechanism/assembly, when provided, can be employed to track the number of times a medical device and/or the surgical instrument assembly 10 is put to use in a surgical procedure and surgical operation to help manage device and instrument life cycle. As described in this patent, the use count mechanism/assembly is designed and constructed to accurately record and increment the uses of the particular device and/or instrument when employed in a surgical reprocessing workflow. The use count mechanism/assembly can be equipped with a non-back drivable function in order to furnish a more reliable, error-proof, and tamper-proof use count. Further, the tensioner or tensioning mechanism/assembly, when provided, serves to furnish a tension relief function in order to compensate and account for thermal expansion stresses that may be experienced amid reprocessing procedures such as in an autoclave machine or during steam sterilization, among other possibilities. A protective cover can be provided as part of the flush port inaccessibility and accessibility mechanism/assembly, when provided, to conceal and hide the flush port when not utilized, and thereby minimize or altogether thwart bioburden infiltration. Lastly, the obsolescence mechanism/assembly, when provided, serves to preclude and prevent unsanctioned and perhaps unsafe re-use of the surgical instrument assembly 10 that would surpass the instrument's intended useful life. Together or individually or in combination, these mechanisms and assemblies facilitate patient safety, instrument sterility, instrument life cycle management, and/or instrument reusability; still, a particular embodiment of the surgical instrument assembly 10 can exhibit one or more or none of these advancements, as well as other advancements not mentioned herein.

Various embodiments of the surgical instrument assembly 10 can have various combinations of the mechanisms and assemblies, and some individual components of the many mechanisms and assemblies may be shared, as will become evident as this description advances. For example, in an embodiment the surgical instrument assembly 10 can only have the use count mechanism/assembly, can only have the obsolescence mechanism/assembly, can only have the tensioner or tensioning mechanism/assembly, or can only have the flush port inaccessibility and accessibility mechanism/assembly. Still, in other embodiments the surgical instrument assembly 10 can have the use count mechanism/assembly and the obsolescence mechanism/assembly; can have the use count mechanism/assembly and the tensioner or tensioning mechanism/assembly; or can have the use count mechanism/assembly and the flush port inaccessibility and accessibility mechanism/assembly. In yet other embodiments, the surgical instrument assembly 10 can have the obsolescence mechanism/assembly and the tensioner or tensioning mechanism/assembly; or can have obsolescence mechanism/assembly and the flush port inaccessibility and accessibility mechanism/assembly. In another embodiment, the surgical instrument assembly 10 can have the tensioner or tensioning mechanism/assembly and the flush port inaccessibility and accessibility mechanism/assembly. In further embodiments, the surgical instrument assembly 10 can have the use count mechanism/assembly, the obsolescence mechanism/assembly, and the tensioner or tensioning mechanism/assembly; can have the use count mechanism/assembly, the obsolescence mechanism/assembly, and the flush port inaccessibility and accessibility mechanism/assembly; can have the use count mechanism/assembly, the tensioner or tensioning mechanism/assembly, and the flush port inaccessibility and accessibility mechanism/assembly; or can have the obsolescence mechanism/assembly, the tensioner or tensioning mechanism/assembly, and the flush port inaccessibility and accessibility mechanism/assembly. Still further, in an embodiment the surgical instrument assembly 10 can have all of the use count mechanism/assembly, the obsolescence mechanism/assembly, the tensioner or tensioning mechanism/assembly, and the flush port inaccessibility and accessibility mechanism/assembly.

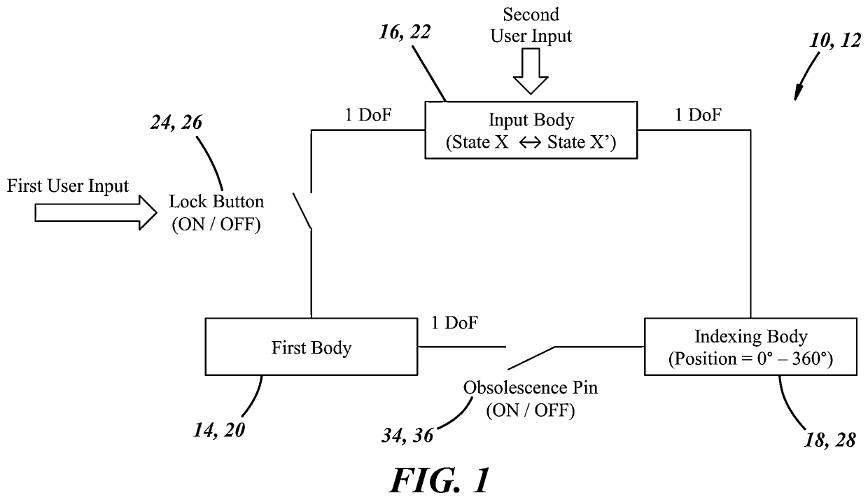

FIG. 1 presents an embodiment of the surgical instrument assembly 10 in block diagrammatic form. Here, the surgical instrument assembly 10 has a first body 14, an input body 16, and an indexing body 18. These bodies can take various forms in various embodiments. Depending on the embodiment and application of the surgical instrument assembly 10, the first body 14, input body 16, and indexing body 18 can each be a single, discrete body or an assembly of multiple discrete bodies joined together in various ways. In the example application of the handheld surgical instrument 12, and as depicted and described elsewhere, the first body 14 can be in the form of a frame body 20. The frame body 20, in general, can serve as a common or reference ground for other mechanisms and assemblies of the surgical instrument assembly 10 (e.g., for the use count mechanism/assembly), per various embodiments. The frame body 20 can be a multi-featured body made-up of many joints between discrete bodies. In the embodiment of the use count mechanism/assembly, the joints are not necessarily limited thereto and rather can include ancillary joints that serve other functions apart from the use count mechanism/assembly. In the application of the handheld surgical instrument 12, the frame body 20 can constitute an intermediate body situated between an input mechanism/assembly thereof such as a handle input (introduced below), and an output mechanism/assembly such as an end effector assembly (introduced below).

Furthermore, the input body 16 is moveable with respect to the first/frame body 14, 20 during use of the surgical instrument assembly 10, and prompts movement of the indexing body 18 during use of the surgical instrument assembly 10. The user of the surgical instrument assembly 10 can interact with the instrument via the input body 16. The input body 16 can have a first position with respect to the first/frame body 14, 20, and can have a second position with respect to the first/frame body 14, 20. The input body 16 can be in the form of a counter ring body 22 or counter knob. In the embodiment of FIG. 1, a single DoF is provided between the first body 14 and input body 16, and between the frame body 20 and counter ring body 22. Accordingly, five DoCs are provided between the first body 14 and input body 16 and between the frame body 20 and counter ring body 22, constraining the bodies together apart from the single DoF. The single DoF, when provided, can be a rotational DoF. The single DoF and single rotational DoF can be the sole degree of freedom established between the first body 14 and input body 16, and between the frame body 20 and counter ring body 22. Here, the input body 16 and counter ring body 22 can have a first discrete rotational position with respect to the first/frame body 14, 20, and can have a second discrete rotational position with respect to the first/frame body 14, 20.

With continued reference to FIG. 1, movement between the first and second discrete rotational positions is rotational movement of the input body 16 and counter ring body 22 with respect to the first body 14 and frame body 20, according to this embodiment. The rotational movement can be a first rotational direction to the first discrete rotational position, and a second, opposite rotational direction to the second discrete rotational position. The first rotational direction can be in the clockwise direction, and the second rotational direction can be in the counterclockwise direction. The first discrete rotational position can constitute an endpoint of rotational movement of the input body 16 and counter ring body 22 with respect to the first body 14 and frame body 20 in that particular direction, and the second discrete rotational position can likewise constitute an endpoint of rotational movement of the input body 16 and counter ring body 22 with respect to the first body 14 and frame body 20 in that particular direction. The first position and first discrete rotational position can constitute a home position, and the second position and second discrete rotational position can constitute an interim position. In the application of the handheld surgical instrument 12, the first position and first discrete rotational position can put the handheld surgical instrument 12 in a use condition and state in which the instrument is ready for use in an MIS procedure (denoted State X in FIG. 1), and the second position and second discrete rotational position can bring the handheld surgical instrument 12 to a non-use or reprocessing-ready or storage condition and state (denoted State X′ in FIG. 1). Further, in an embodiment, the input body 16 and counter ring body 22 can have a rotational joint with respect to the first/frame body 14, 20; here, a DoF axis of the rotational joint can have a concentric arrangement and relationship with a shaft axis or x-axis (FIG. 8) in the application of the handheld surgical instrument 12. The input body 16 and counter ring body 22 would then move with respect to the first/frame body 14, 20 and about the shaft axis and x-axis.

Furthermore, a second user input or user input action incites movement of the input body 16 with respect to the first body 14, and incites movement of the counter ring body 22 with respect to the frame body 20. In a medical setting, the second user input can be carried out by a nurse or personnel of a central sterile processing department (CSPD), as examples. In an embodiment, the second user input can be physical exertion from a user's hand (e.g., user's thumb and index finger) that moves the input body 16 with respect to the first body 14, and that moves the counter ring body 22 with respect to the frame body 20. The second user input can be rotational user input, per an embodiment. Further, in an embodiment, the second user input can be an alternative form of input that need not necessarily involve the user of the larger assembly or instrument; this applies to user inputs for the surgical instrument assembly 10 described elsewhere in this patent. For instance, the input can be generated by other mechanisms and assemblies that are disassociated from the user. As an example, a thermally activated switch can serve to impart the input action that incites movement of the input body 16 with respect to the first body 14, and that incites movement of the counter ring body 22 with respect to the frame body 20. The thermally activated switch can employ a bimetallic strip or nitinol or some other component or technique. Further, a temperature-deformable body can serve to impart the input action that incites movement of the input body 16 with respect to the first body 14, and that incites movement of the counter ring body 22 with respect to the frame body 20. In this example, upon experiencing an increase in temperature, the temperature-deformable body could prompt such movement itself or could permit such movement. Permitting such movement could involve relief of a movement constraint otherwise established between the input body 16 and first body 14, and between the counter ring body 22 and frame body 20 (e.g., a biasing exertion could, upon relief of the constraint, impart the relative movement). In examples in which initiation and prompting occurs via an elevation in temperature, the temperature increase can result from processing within an autoclave machine. In other examples, the input action that incites movement could be a consequence of conditions that the surgical instrument assembly 10 experiences. In the application of the handheld surgical instrument 12, the conditions could be abuse loads, inappropriate storage conditions, or other unwanted conditions. Moreover, the input body 16 and the associated input action could be various components, assemblies, and/or conditions that are suitable for inciting movement of the input body 16 with respect to the first body 14, and inciting movement of the counter ring body 22 with respect to the frame body 20.

With continued reference to FIG. 1, in this embodiment a retention body 24 is situated between the first/frame body 14, 20 and the input body 16 and counter ring body 22. The retention body 24 serves to temporarily constrain the single DoF and single rotational DoF provided between the bodies, and retains the input body 16 with respect to the first body 14 and the counter ring body 22 with respect to the frame body 20. When unactuated, the retention body 24 can maintain the input body 16 and counter ring body 22 in its first position and first discrete rotational position and/or in its second position and second discrete rotational position. The input body 16 and counter ring body 22 are prevented and blocked from movement via the retention body 24. And conversely, when actuated, the input body 16 and counter ring body 22 is released and can move with respect to the first/frame body 14, 20.

In the embodiment of FIG. 1, the retention body 24 is in the form of a lock button 26. The lock button 26 can serve to preclude unintentional and unwanted activation and incitement of movement of the input body 16 and counter ring body 22. In an embodiment with the use count mechanism/assembly, the lock button 26 precludes and prevents unintentional incrementation and advancement of the use count; in an embodiment with the flush port inaccessibility and accessibility mechanism/assembly, the lock button 26 precludes and prevents unintentional revelation of the flush port otherwise concealed via a flush port cover body (introduced below); and in an embodiment with the tensioner or tensioning mechanism/assembly, the lock button 26 precludes and prevents unintentional tension relief of the associated transmission cables. The lock button 26 has an ON state and position, and an OFF state and position. The lock button 26 can be spring-loaded, or biased in other ways such as via a compliant connector, to retain the input body 16 with respect to the first body 14 and the counter ring body 22 with respect to the frame body 20. When retained, the lock button 26 can engage with the input body 16 and the counter ring body 22; and when engaged therewith, the lock button 26 constrains the single DoF and single rotational DoF provided between the bodies. Further, in the embodiment and application of the handheld surgical instrument 12, the lock button 26 can be located at the frame body 20 and can be carried thereby. The lock button 26 can be equipped with the frame body 20 by a prismatic joint, per an embodiment. A single DoF can be provided between the lock button 26 and frame body 20; per an embodiment, the single DoF is a single translational DoF. Still, other DoFs and more than a single DoF can be provided between the lock button 26 and frame body 20 in other embodiments. In a further embodiment, the lock button 26 could have an interlock that allows/disallows activation based on alternate electromechanical methods such as a proximity sensor within a healthcare facility; this would furnish additional safety measures.

In this embodiment a first user input or user input action actuates the lock button 26. In a medical setting, the first user input can be carried out by a nurse or personnel of the central sterile processing department (CSPD), as examples. Actuation can be effected via a user pressing the lock button 26 inward toward the frame body 20; still, other input actions and other ways of actuating the lock button 26 are possible in other embodiments. By pressing the lock button 26, according to this embodiment, the lock button 26 moves and is displaced radially inboard with respect to the frame body 20. Here, there is concurrent relative movement between the lock button 26 and counter ring body 22 upon its pressing, in addition to that between the lock button 26 and frame body 20. Actuation brings the lock button 26 from the ON state and to the OFF state. When ON, an interlock is activated between the lock button 26 and counter ring body 22, and when OFF, the interlock between the bodies is deactivated and removed. The interlock can be activated when the surgical instrument assembly 10 and handheld surgical instrument 12 is in the use state, as well as when the surgical instrument assembly 10 and handheld surgical instrument 12 is in the non-use state. The interlock can be effected via a mechanical and structural interference between the lock button 26 and the counter ring body 22, effectively joining the bodies together and providing a rigid joint therebetween that lacks degrees of freedom. Moreover, the lock button 26 can have an up position and locked state, and conversely can have a down position and unlocked state. The lock button 26 can have an unpressed position and locked state, and conversely can have a depressed position and locked state. The lock button 26 can be biased and urged toward its ON and locked state. Furthermore, in an embodiment that employs the tensioner or tensioning mechanism/assembly, the lock button 26 can hold the mechanism/assembly in a tensioned or un-tensioned state, depending on the implementation; still, a detent mechanism or a non-back drivable function or other retention body can hold the mechanism/assembly in the tensioned or un-tensioned state, per alternative embodiments.

In an alternative embodiment, the lock button 26 can incorporate a thermally-activated provision to help preclude excessive tensioning of transmission cables when they are under thermal expansion stress and when other components of potentially dissimilar material compositions are also under thermal expansion stress, further ensuring safe operating conditions for use of the surgical instrument assembly 10. The thermally-activated provision can take the form of a shape memory alloy (SMA) component, such as a thermal switch. The SMA component and its functionality can supplement user input action, or can altogether supersede it, rendering user input unneeded. In one implementation of this embodiment, the SMA component maintains the locked state and prevents movement of the input body 16 and counter ring body 22 when experiencing increased temperatures, thereby precluding placement of the transmission cables in a tensioned state that could otherwise over-tension them. Furthermore, and like other bodies described in this patent, the retention body 24 and lock body 26 need not be provided in a particular embodiment of the surgical instrument assembly 10.

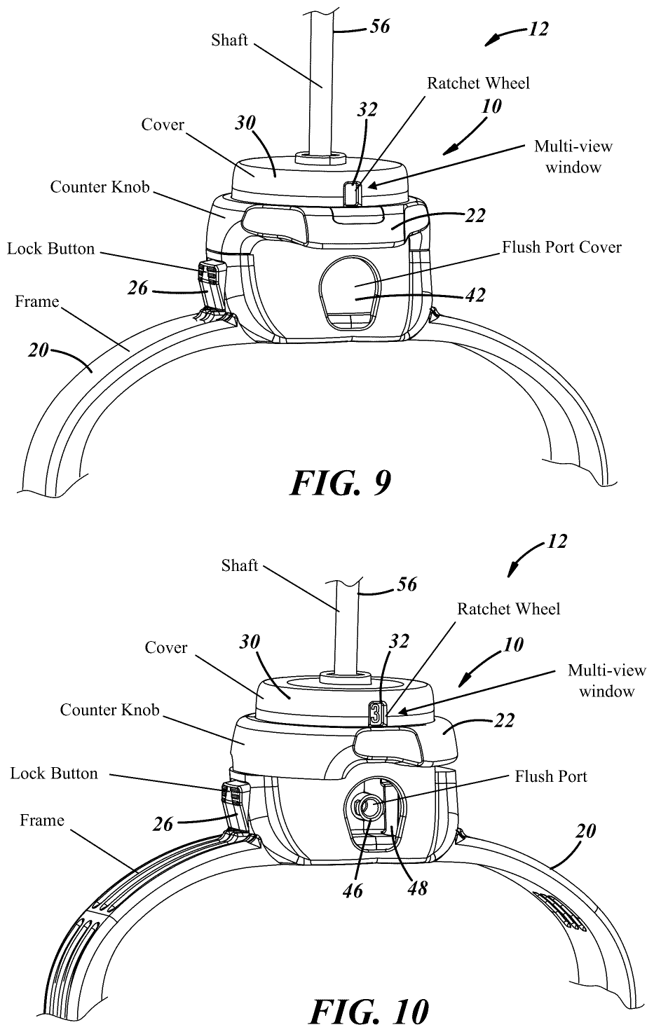

Still referring to FIG. 1, the indexing body 18 is moveable with respect to the first/frame body 14, 20 during use of the surgical instrument assembly 10, and is caused to move via movement of the input body 16 and counter ring body 22. The indexing body 18 is a component of the use count mechanism/assembly. The indexing body 18 can be moved to a multitude of indexed positions relative to the first/frame body 14, 20. The multitude of indexed positions can include a final indexed position, as set forth below in greater detail, the attainment of which can serve to fully constrain movement of the input body 16 and counter ring body 22 with respect to the first/frame body 14, 20, and/or can serve to fully constrain movement of the indexing body 18 with respect to the first/frame body 14, 20. The indexing body 18 can be in the form of a ratchet wheel body 28 or ratchet wheel, per an embodiment. The ratchet wheel body 28 can be ring-shaped. In the embodiment of FIG. 1, a single DoF is provided between the input body 16 and counter ring body 22 and the indexing body 18 and ratchet wheel body 28. The single DoF can be a single rotational DoF, per an embodiment. Further, in this embodiment, a single DoF can be provided between the indexing body 18 and ratchet wheel body 28 and the first/frame body 14, 20; per an embodiment, the single DoF is a single rotational DoF. The indexing body 18 and ratchet wheel body 28 can have an interior location in the surgical instrument assembly 10, and can be situated radially-inboard of the input body 16 and of the counter ring body 22. The ratchet wheel body 28 can be carried by the counter ring body 22. In the embodiment and application of the handheld surgical instrument 12, and with momentary reference to FIG. 9, an external cover body 30 partially conceals the ratchet wheel body 28 in assembly. A viewing window 32 residing and defined in the external cover body 30 reveals indicia of the ratchet wheel 28, as described below in greater detail.

In general, during use of the surgical instrument assembly 10 and amid performance of its function, discrete indexed positions of the multitude of indexed positions can serve to bring discrete indicium of the ratchet wheel body 28 in alignment with the viewing window 32 for display and viewing by the user of the instrument. Such alignment can occur with each rotational position of the ratchet wheel body 28 relative to the first/frame body 14, 20 and to the external cover body 30. Discrete indexed positions can be discrete rotational positions. The indicia can be a set of symbols, colors, numbers, and/or other representations that indicate the immediate condition and state of the instrument and/or end-of-life information of the instrument, among other possible representations and information conveyed to the user. The indicia can be a use count indicia, per an embodiment. By way of this functionality, each rotation of the input body 16 and counter ring body 22 can serve to increment a use count of the surgical instrument assembly 10, per an embodiment, conveying the end-of-life information, and hence allowing the user to track the usage and condition of the instrument. Moreover, functioning of the indexing body 18 and the ratchet wheel body 28 can be coupled with, and linked to, functioning of the counter ring body 22, as will become more apparent as this description proceeds.

With continued reference to FIG. 1, in an embodiment the surgical instrument assembly 10 can also include an obsolescence body 34. The obsolescence body 34, when provided, is a component of the obsolescence mechanism/assembly. The obsolescence mechanism/assembly and the obsolescence body 34, when triggered and activated, serve to preclude and prevent further usage of the surgical instrument assembly 10 by the user. The surgical instrument assembly 10 can be permanently maintained in its non-use state, for example per an embodiment, via the obsolescence mechanism/assembly and obsolescence body 34, and thereby incapable of additional proper use and functioning. Activation of the obsolescence mechanism/assembly and obsolescence body 34 can mark and establish the end-of-life stage and state of the surgical instrument assembly 10, and cessation of its subsequent and continued employment and use in MIS procedures, for instance, in the embodiment and application of the handheld surgical instrument 12. Ultimately, the obsolescence mechanism/assembly and obsolescence body 34 can facilitate patient safety and user safety. In the embodiment of FIG. 1, obsolescence of the surgical instrument assembly 10 can occur after the total number of intended uses of the instrument has been reached and exhausted, among other possibilities. Furthermore, in an embodiment, the obsolescence mechanism/assembly and the obsolescence body 34 can be incorporated in the use count mechanism/assembly.

The obsolescence body 34 is moveable with respect to the first/frame body 14, 20 during use of the surgical instrument assembly 10 and when triggered and activated. In an embodiment, the obsolescence body 34 can be situated at the indexing body 18 and at the ratchet wheel body 28, and can be carried by the indexing body 18 and by the ratchet wheel body 28. The obsolescence body 34 can be in the form of a lock body 36 or obsolescence lock pin, per an embodiment. In the embodiment of FIG. 1, a single DoF is provided between the obsolescence body 34 and lock body 36 and the indexing body 18 and ratchet wheel body 28. The single DoF can be a single translational DoF, per an embodiment. A prismatic joint can be established between obsolescence body 34 and lock body 36 and the indexing body 18 and ratchet wheel body 28. Further, the obsolescence body 34 and lock body 36 has an ON state and position, and has an OFF state and position. In the ON state, the obsolescence body 34 and lock body 36 is deployed, and in the OFF state, the obsolescence body 34 and lock body 36 is retracted. The obsolescence body 34 and lock body 36 can be spring-loaded or biased in other ways such as via a compliant connector to its deployed state. The ON state, per an embodiment, is only established when the indexing body 18 and ratchet wheel body 28 is brought to the final indexed position. The ON state is also referred to as the obsolescence state of the surgical instrument assembly 10, or final state or terminal state. The obsolescence state constitutes the end-of-life state of the surgical instrument assembly 10.

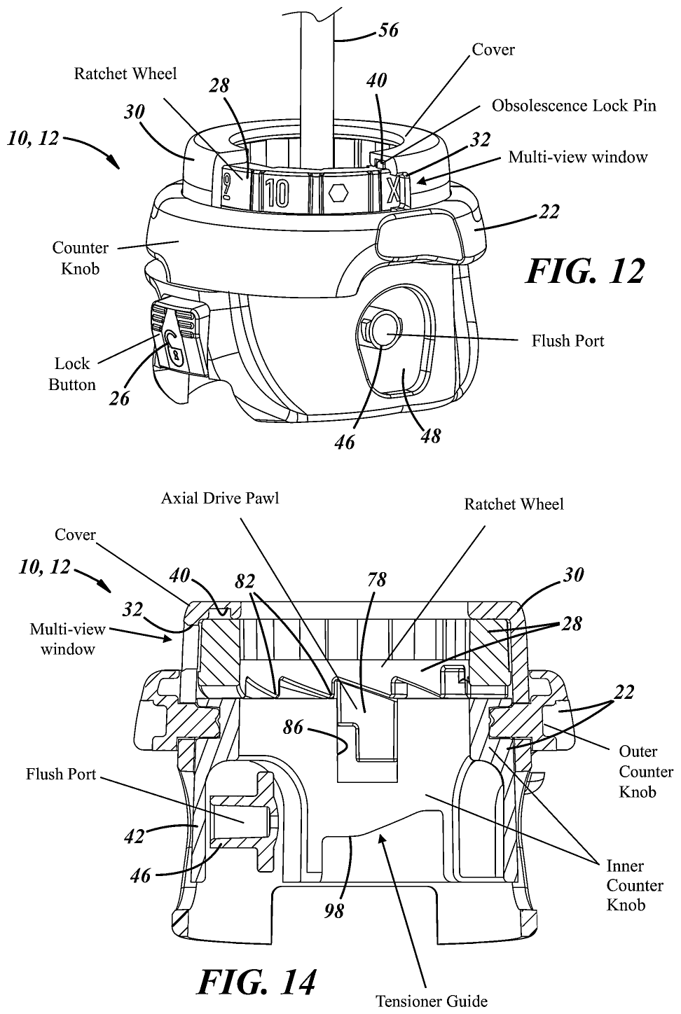

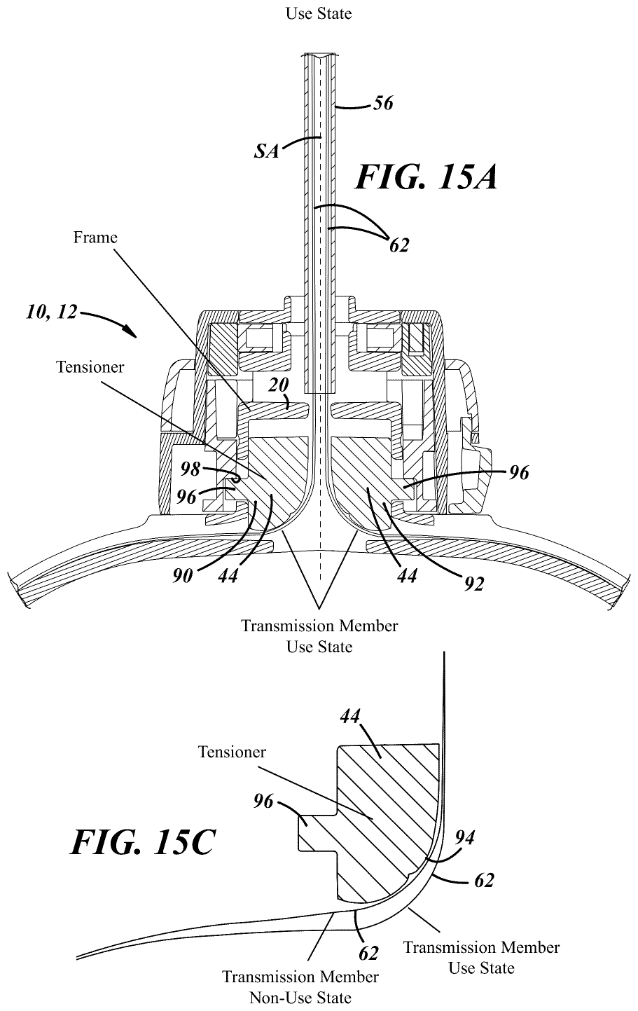

In the embodiment and application of the handheld surgical instrument 12, and with reference now to FIGS. 11-14, the lock body 36 is seated within a pocket 38 residing and defined in the ratchet wheel body 28. The pocket 38 is also referred to as an obsolescence lock pin guide. The pocket 38 has an open top end. In the pocket 38, movement of the lock body 36 is constrained and restricted to axial movement in a single direction. The lock body 36 moves and revolves with movement of the ratchet wheel body 28 during use of the surgical instrument assembly 10. A spring or other biasing member or compliant connector urges and biases the lock body 36 axially outboard and upward and toward the external cover body 30. The external cover body 30 can have a fixed connection with the frame body 20 and can be fully constrained with respect to each other; in this regard, from a kinematics perspective, the external cover body 30 and frame body 20 can constitute a single body. In this embodiment, when the indexing body 18 and ratchet wheel body 28 is in its indexed positions prior to the final indexed position (i.e., pre- and non-final indexed positions), the lock body 36 is prevented and blocked from movement out of the pocket 38 via surface-to-surface abutment with the external cover body 30. The lock body 36 remains seated in, and is constrained within, the pocket 38. The lock body 36 remains retracted. Furthermore, in this embodiment, the lock body 36 deploys and is urged to project partly out of the pocket 38 only when the indexing body 18 and ratchet wheel body 28 is brought to the final indexed position. Here, the lock body 36 is brought into alignment with a recess 40. The recess 40 is also referred to as an obsolescence lock pin hole. In the embodiment of FIGS. 11-14, the recess 40 resides and is defined in the external cover body 30 and, particularly, at an underside thereof. Still, in other embodiments the recess 40 could have other locations and can reside in other components of the surgical instrument assembly 10 such as at the frame body 20; in these alternatives, the lock body 36 could also have other locations and be carried by other components and have other arrangements. When in-line and aligned, the lock body 36 projects out of the pocket 38 and is urged into insertion and reception in the recess 40. The lock body 36 and recess 40 engage each other and maintain their engagement. An interlock is hence established between the components via the lock body 36 and recess 40 inter-engagement. As a consequence, the ratchet wheel body 28 is fully constrained with the external cover body 30 and unable to move with respect thereto and unable to move with respect to the frame body 20. The constraint can be a full rotational constraint therebetween, and can be permanently made and established, per an embodiment. By way of this constraint, the counter ring body 22 is also fully constrained with respect to the frame body 20. Moreover, functioning of the obsolescence body 34 and the lock body 36 can be coupled with, and linked to, functioning of the counter ring body 22, as will become more apparent as this description proceeds.

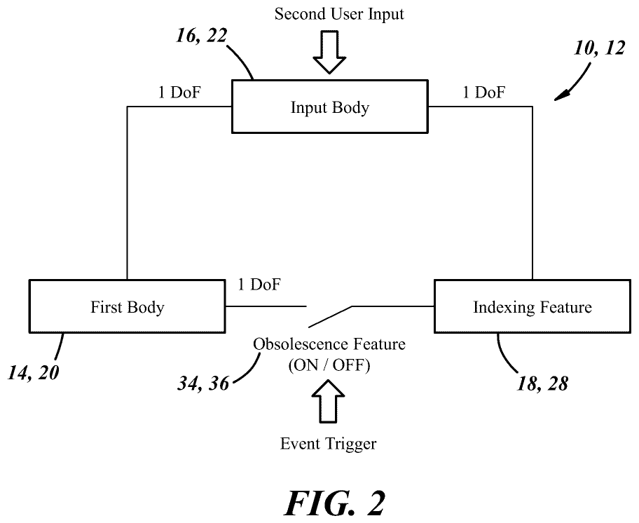

FIG. 2 presents another embodiment of the surgical instrument assembly 10 in block diagrammatic form. In this embodiment, the obsolescence mechanism/assembly and obsolescence body 34 can be triggered and activated in other ways according to various embodiments. Indeed, triggering and activation can be disassociated and divorced from user input action and/or from the use count mechanism/assembly. In the embodiment of FIG. 2, for example, an event trigger can cause activation of the obsolescence mechanism/assembly and obsolescence body 34. The event trigger can be a system level adverse event, per an embodiment. Examples according to various embodiments include one or more of the following: i) excessive tensioning of transmission cables; ii) abuse loads experienced through a shaft body of the handheld surgical instrument 12 and transmission cable system; iii) excessive cautery level in an embodiment of the handheld surgical instrument 12 in which a cauterize mechanism and assembly and cauterize capabilities are furnished; iv) a temperature of the surgical instrument assembly 10 and/or of the handheld surgical instrument 12 or component or portion thereof becomes excessively hot; v) storage conditions of the surgical instrument assembly 10 and/or of the handheld surgical instrument 12 that are unsuitable such as environmental conditions with excessive humidity or excessive temperatures; vi) the surgical instrument assembly 10 and/or handheld surgical instrument 12 is inadvertently dropped or otherwise grossly mishandled; vii) an expiration date of the surgical instrument assembly 10 and/or handheld surgical instrument 12 is surpassed; viii) excessive reprocessing procedures and cycles such as cycles in an autoclave machine or steam sterilization machine (this can be independent of the number of uses of the surgical instrument assembly 10 and/or of the handheld surgical instrument 12); and/or ix) use cycles of the surgical instrument assembly 10 and/or of the handheld surgical instrument 12, jaw actuation of the surgical instrument assembly 10 and/or of the handheld surgical instrument 12, and/or total number of articulation cycles of the surgical instrument assembly 10 and/or of the handheld surgical instrument 12; still, other event triggers are possible in other embodiments. Moreover, in these embodiments of the obsolescence mechanism/assembly and obsolescence body 34, as well as in other embodiments, the obsolescence body 34 could take other forms apart from the lock body 36 and obsolescence lock pin, and there could be multiple obsolescence bodies that interact with other components such as the frame body in independent ways relative to one another.

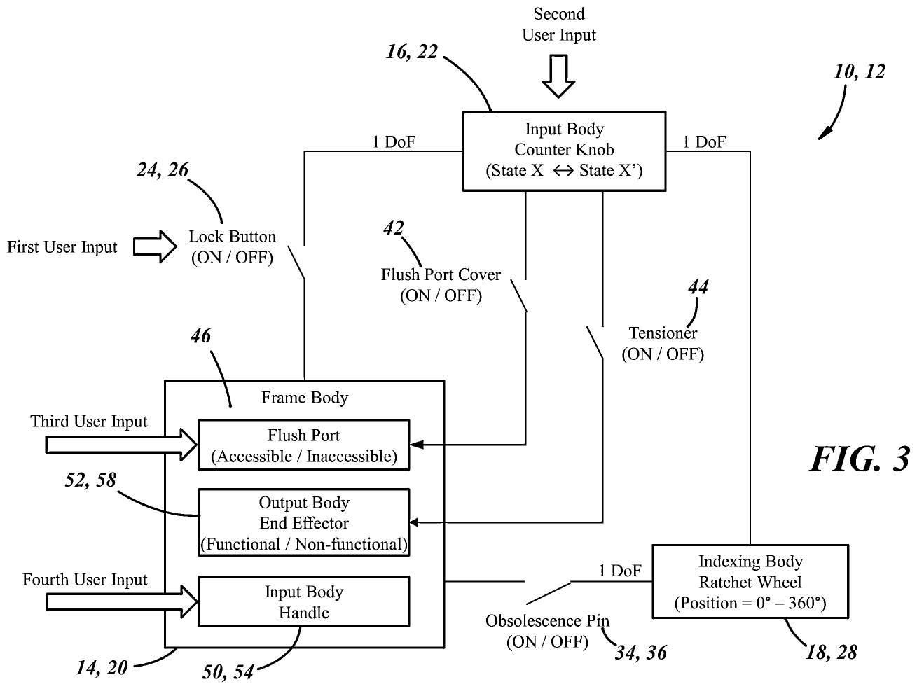

FIG. 3 presents another embodiment of the surgical instrument assembly 10 in block diagrammatic form. In this embodiment, the surgical instrument assembly 10 further includes a flush port cover body or just cover body 42, and a tensioner or tensioning body 44. The bodies can take various forms in various embodiments. Depending on the embodiment and application of the surgical instrument assembly 10, the cover body 42 and tensioning body 44 can each be a single, discrete body or an assembly of multiple discrete bodies joined together in various ways.

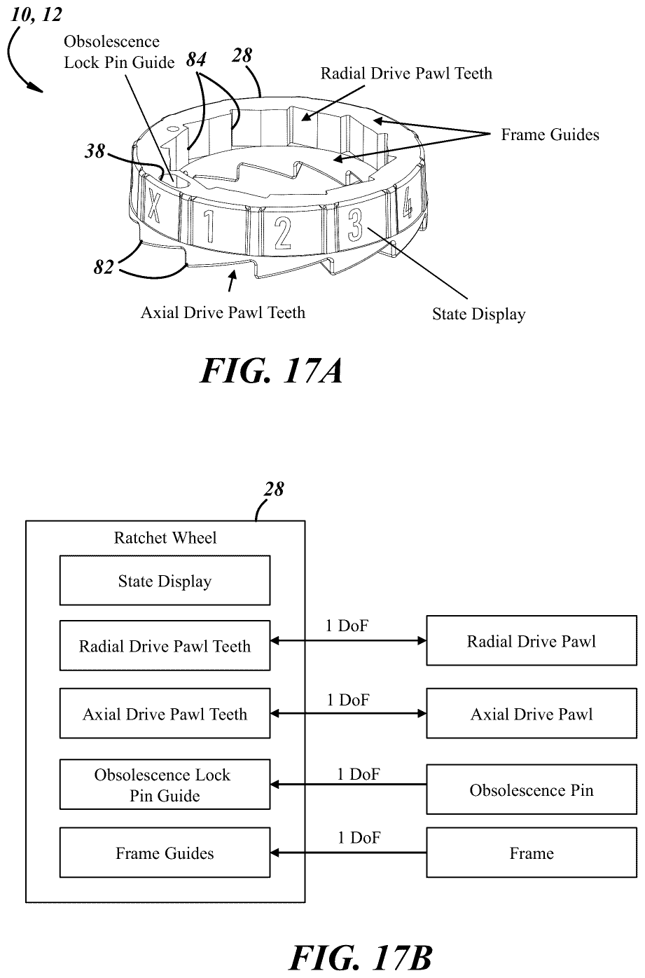

In an embodiment, the cover body 42 is moveable with respect to the first/frame body 14, 20 during use of the surgical instrument assembly 10, and is caused to move via movement of the input body 16 and counter ring body 22. The cover body 42 is a component of the flush port inaccessibility and accessibility mechanism/assembly. During use of the surgical instrument assembly 10, when the input body 16 and counter ring body 22 is in its first position, the cover body 42 renders and makes a flush port 46 of the surgical instrument assembly 10 inaccessible. Conversely, when the input body 16 and counter ring body 22 is in its second position, the cover body 42 renders and makes the flush port 46 accessible. When inaccessible, the cover body 42 physically blocks and conceals and hides the flush port 46. And when accessible, the blocking and concealment via the cover body 42 is removed and absent, and the flush port 46 is available for a flushing procedure. Further, the cover body 42 has an ON state and position, and has an OFF state and position. In the ON state, the flush port 46 is accessible, and in the OFF state, the flush port 46 is inaccessible via the cover body 42. The cover body 42 can have a first position with respect to the first/frame body 14, 20, and can have a second position with respect to the first/frame body 14, 20. The first and second positions can be first and second discrete rotational positions with respect to the first/frame body 14, 20. The first position can constitute the OFF state, and the second position can constitute the ON state. In the embodiment and application of the handheld surgical instrument 12, the flush port 46 can exhibit fluid communication with an accompanying shaft body for flushing procedures thereof. With reference to FIG. 16a, in this embodiment the cover body 42 is integral with, and a unitary extension of, the counter ring body 22. The cover body 42 and its functionality can be constituted by, and be carried out via, a wall portion of the counter ring body 22 as presented in the figures, a shield body, or some other type of barrier body. The cover body 42 is fully constrained with the counter ring body 22. A flush port opening 48 resides and is defined in a wall of the counter ring body 22 beside the cover body 42. The flush port 46 is revealed and accessible via the flush port opening 48. Since, according to this embodiment, the cover body 42 is integral and unitary with the counter ring body 22, movements and positions of the bodies and components correspond with each other; that is, for instance, the second user input to the counter ring body 22 concurrently incites movement of the cover body 42. Functioning of the cover body 42 can be coupled with, and linked to, functioning of the counter ring body 22. Further, with reference again to FIG. 3, a third user input or user input action is directed at the flush port 46 when it is accessible. The third user input can occur during the non-use and reprocessing-ready state of the surgical instrument assembly 10, and can involve personnel of the central sterile processing department (CSPD) attaching a hose of the flush port 46 for performance of a flushing procedure. Still, in other embodiments, the flush port could be a separate body that connects with the surgical instrument assembly such as with the frame body.

With continued reference to FIG. 3, in this embodiment, the tensioning body 44 is moveable with respect to the first/frame body 14, 20 during use of the surgical instrument assembly 10, and is caused to move via movement of the input body 16 and counter ring body 22. The tensioning body 44 is a component of the tensioner or tensioning mechanism/assembly. The tensioner or tensioning mechanism/assembly and tensioning body 44 serve to alter and adjust the tension of one or more transmission cables of the surgical instrument assembly 10 and/or of the handheld surgical instrument 12. The tensioner or tensioning mechanism/assembly is also referred to as the articulation cable tensioner or tensioning mechanism/assembly, the transmission system tensioner or tensioning mechanism/assembly, the cable tensioner or tensioning mechanism/assembly, and/or the end effector transmission cable tensioner or tensioning mechanism/assembly. Having the tensioner or tensioning mechanism/assembly and tensioning body 44 has proven beneficial, per an embodiment, by furnishing tension variation capabilities for optimization of desired target tensions, and minimizing or altogether sidestepping harm that could otherwise arise due to over-stress and excessive tension of the one or more transmission cables, among other possible benefits.

During use of the surgical instrument assembly 10, when the input body 16 and counter ring body 22 is in its first position, a first tension and first tension magnitude is provided to the one or more transmission cables of the surgical instrument assembly 10 and/or of the handheld surgical instrument 12 via the tensioning body 44. Conversely, when the input body 16 and counter ring body 22 is in its second position, a second tension and second tension magnitude is provided to the one or more transmission cables of the surgical instrument assembly 10 and/or of the handheld surgical instrument 12 via the tensioning body 44. The first tension has a first value that is suitable for proper functioning and output and motion transmission from an input body 50 and to an output body 52 of the surgical instrument assembly 10 and/or of the handheld surgical instrument 12 via the one or more transmission cables. The second tension, on the other hand, has a second value that is unsuitable for proper functioning and output and motion transmission from the input body 50 and to the output body 52 via the one or more transmission cables. The second tension has a value that is less than that of the first tension. The one or more transmission cables exhibit a suitably tightened state when subject to the first tension, and, conversely, exhibit a comparatively loosened state when subject to the second tension. Further, the tensioning body 44 has an ON state and position, and has an OFF state and position. In the ON state, the first tension is provided to the one or more transmission cables; and in the OFF state, the second tension is provided to the one or more transmission cables. In this embodiment, a single DoF is provided between the tensioning body 44 and the input body 16 and counter ring body 22; the single DoF can be in the form of a screw pair, per an embodiment. Further, in this embodiment a single DoF is provided between the tensioning body 44 and the first/frame body 14, 20; the single DoF can be a single translational DoF, per an embodiment. Moreover, functioning of the tensioning body 44 can be coupled with, and linked to, functioning of the counter ring body 22, as will be described in greater detail below.

With continued reference to FIG. 3, the input body 50 can be in the form of a handle assembly or body 54. The input body 50 and handle body 54 can take various forms in various embodiments. Depending on the embodiment and application of the surgical instrument assembly 10, the input/handle body 50, 54 can be a single, discrete body or an assembly of multiple discrete bodies joined together in various ways. The handle body 54 can be a component of the handheld surgical instrument 12 (FIG. 8). The handle body 54 can constitute a user interface of the handheld surgical instrument 12. A fourth user input or user input action can be provided at the input/handle body 50, 54 by the user of the surgical instrument assembly 10. In the embodiment and application of the handheld surgical instrument 12, a surgeon or other user of the handheld surgical instrument 12 exerts the fourth user input. In various embodiments, the fourth user input can be a manipulation of the handle body 54 such as articulation input motions, rigid body motions, and/or roll input motions, among other possible user inputs and user input actions. The articulation input motions can be pitch articulation input motions, yaw articulation input motions, or both pitch and yaw articulation input motions, per various embodiments. As shown in FIG. 8, the rigid body motions can be along an x-axis or shaft axis SA, can be along a y-axis (e.g., right and left movements), and/or can be along a z-axis (e.g., up and down movements). The shaft axis SA is established by an elongated extent of a shaft body 56 of the handheld surgical instrument 12. The shaft body 56 can be rigidly connected to the frame body 20, and can itself be rigid in construction or compliant in bending, according to various embodiments. The roll input motions are about the shaft axis SA. At least some of the input motions are transmitted to the output body 52. Furthermore, in the embodiment and application of the handheld surgical instrument 12, an articulation input joint can be established between the handle body 54 and frame body 20 for capturing articulation input motions at the handle body 54 and transmitting the motions to the output body 52. Example handle bodies, shaft bodies, and articulation input joints are depicted and described in U.S. Pat. No. 11,950,966 issued on Apr. 9, 2024 and owned by present applicant FlexDex, Inc., the contents of which are hereby incorporated by reference in their entirety. Further examples of handle bodies, shaft bodies, and articulation input joints are depicted and described in U.S. Patent Application Publication No. 2023/0040475 published on Feb. 9, 2023 and owned by present applicant FlexDex, Inc., the contents of which are hereby incorporated by reference in their entirety.

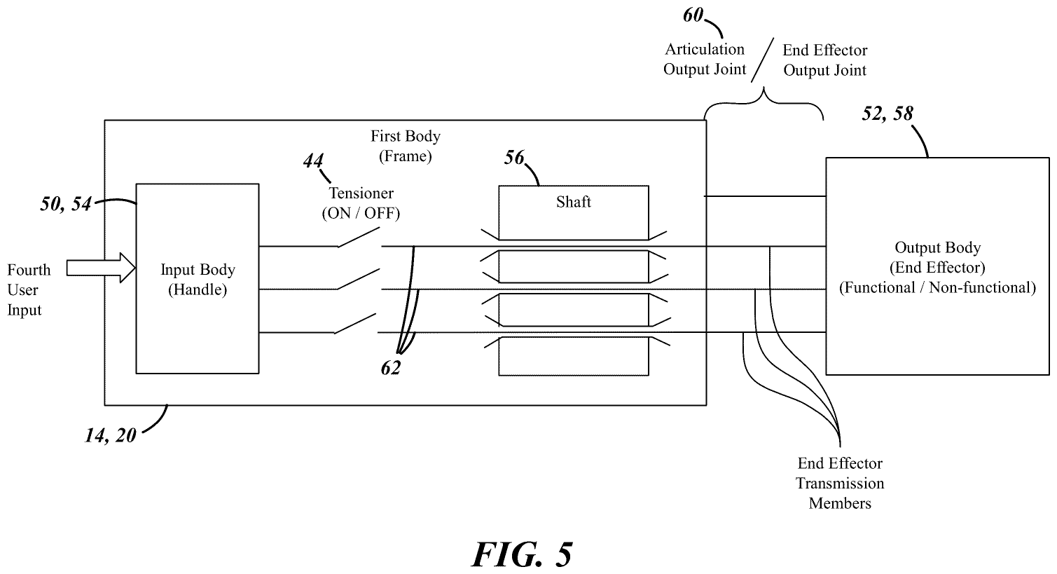

With reference to FIG. 3, the output body 52 can be in the form of an end effector assembly or body 58. The output body 52 and end effector body 58 can take various forms in various embodiments. Depending on the embodiment and application of the surgical instrument assembly 10, the output/end effector body 52, 58 can be a single, discrete body or an assembly of multiple discrete bodies joined together in various ways. The end effector body 58 can be a component of the handheld surgical instrument 12 (FIG. 8). The end effector body 58 can be coupled to a distal and terminal end of the shaft body 56 by way of an articulation output joint 60 (FIG. 5). In various embodiments, the end effector body 58 can include various designs, constructions, and components that perform various functions. For example, the end effector body 58 can have jaws that open and close for grasping objects, or can have scissor blades that open and close for severing objects, among other possibilities. Furthermore, the end effector body 58 provides rigid termination and mounting points for the one or more transmission cables, according to an embodiment. Example end effector bodies are depicted and described in U.S. Pat. No. 11,950,966 issued on Apr. 9, 2024 and owned by present applicant FlexDex, Inc., the contents of which are hereby incorporated by reference in their entirety. Further examples of end effector bodies are depicted and described in U.S. Patent Application Publication No. 2023/0040475 published on Feb. 9, 2023 and owned by present applicant FlexDex, Inc., the contents of which are hereby incorporated by reference in their entirety.

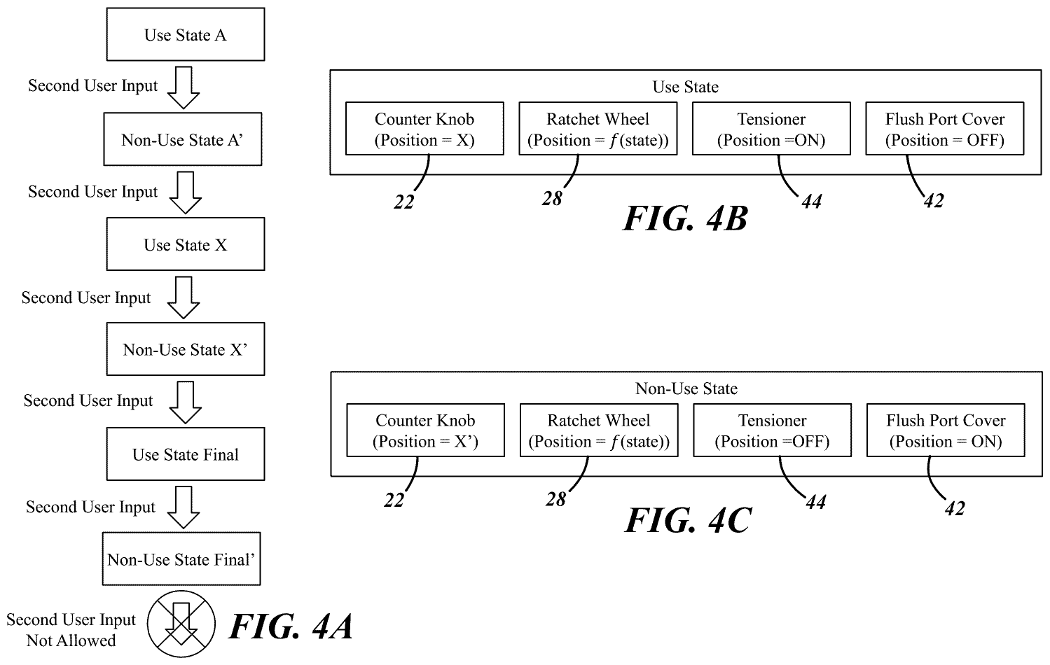

FIG. 4a presents an embodiment of use and non-use states and conditions of the surgical instrument assembly 10 and sequence and transitions thereof to a final non-use state or condition. In this embodiment, the surgical instrument assembly 10 shifts from an initial use state A and to an initial non-use state A′. In an example, after a surgical procedure is performed while the surgical instrument assembly 10 is in the initial use state A, the surgical instrument assembly 10 can be brought to the initial non-use state A′ for reprocessing and/or storage, readying it for later use in a later surgical procedure. The second user input and user input action, per an embodiment, prompts transition and shifting from the initial use state A to the initial non-use state A′, and, contrarily, prompts transition and shifting from the initial non-use state A′ and to a subsequent, intervening use state X. From the use state to the non-use state, per an embodiment, the second user input serves to rotate the input body 16 and counter ring body 22 with respect to the first/frame body 14, 20 in the first rotational direction to the first discrete rotational position. Conversely, from the non-use state to the use state, per this embodiment, the second user input serves to rotate the input body 16 and counter ring body 22 with respect to the first/frame body 14, 20 in the second rotational direction to the second discrete rotational position. Such rotations drive corresponding movements and rotations of the indexing body 18 and ratchet wheel body 28.

Further, a subsequent second user input and user input action prompts transition and shifting to a subsequent, intervening non-use state X′. The quantity of intervening use and non-use states X, X′ can vary in various embodiments. In an example embodiment, the total number of use and non-use states of the surgical instrument assembly 10 can be ten (i.e., ten use states and ten non-use states); still, the number could be more or less in other embodiments. However many, once the final non-use state is reached and established, the second user input action is no longer effective and the surgical instrument assembly 10 cannot be transitioned and shifted out of the final non-use state. The surgical instrument assembly 10 remains in the final non-use state. Furthermore, in the embodiment of the obsolescence mechanism/assembly, the obsolescence body 34 can be triggered and activated at the final non-use state, which can be the final indexed position, per an embodiment. This constitutes the obsolescence state of the surgical instrument assembly 10. Moreover, in the embodiment in which the obsolescence mechanism/assembly and obsolescence body 34 can be triggered and activated via the event trigger such as the system level adverse event, such event occurrence and concomitant triggering can halt and stymie further transition at any of the intervening states—or even at the initial states—and prior to reaching the final non-use state.

FIG. 4b presents an embodiment of the use state and condition of the surgical instrument assembly 10 and of the handheld surgical instrument 12. The use state here can be any of the initial use state A and/or intervening use states X and/or final use state, as set forth in FIG. 4a. In this embodiment of the use state, the input body 16 and counter ring body 22 can be in its first position and first discrete rotational position. And the indexing body 18 and ratchet wheel body 28 can be in one of its discrete indexed positions and discrete rotational positions. Further, in the use state according to this embodiment, the tensioning body 44 can be in its first position and in its ON state, and the cover body 42 can be in its first position and in its OFF state. Moreover, in the use state, the first tension is provided to the one or more transmission cables and the flush port 46 is inaccessible. The lock body 36, if provided, remains retracted. The output/end effector body 52, 58 is in a functional state in the use state.

FIG. 4c presents an embodiment of the non-use state and condition of the surgical instrument assembly 10 and of the handheld surgical instrument 12. The non-use state here can be any of the initial non-use state A′ and/or intervening non-use states X′ and/or final non-use state, as set forth in FIG. 4a. In this embodiment of the non-use state, the input body 16 and counter ring body 22 can be in its second position and second discrete rotational position. And the indexing body 18 and ratchet wheel body 28 can be in one of its discrete indexed positions and discrete rotational positions. Further, in the non-use state according to this embodiment, the tensioning body 44 can be in its second position and in its OFF state, and the cover body 42 can be in its second position and in its ON state. Moreover, in the non-use state, the second tension is provided to the one or more transmission cables and the flush port 46 is accessible. The output/end effector body 52, 58 is in a non-functional state in the non-use state.

FIG. 5 presents an embodiment of the surgical instrument assembly 10 and of the handheld surgical instrument 12 with the tensioner or tensioning mechanism/assembly. Here, a multitude of transmission cables 62 extend between the input/handle body 50, 54 and the output/end effector body 52, 58. The transmission cables 62 can have fixed joints where they terminate at the input/handle body 50, 54 and at the output/end effector body 52, 58. Motions, input, and other functioning at the input/handle body 50, 54 can be transmitted to the output/end effector body 52, 58 via the transmission cables 62. In this embodiment of FIG. 5, the transmission cables 62 are routed and extend through the shaft body 56. The transmission cables 62 can be referred to as transmission members, control cables, elongate transmission members, articulation cables, actuation cables, or simply cables. In various embodiments, the transmission cables 62 can be stainless-steel braided wire ropes that act as a two-force tension member, may not support compressive loads, and may not transmit torque. The transmission cables 62, individually, can only transmit one energy input from an input to an output body. The transmission cables 62 transmit energy vis translational motion. Further, the transmission cables 62 can be routed via routing features that are integral to the bodies through which they are routed. The transmission cables 62 can slide or roll over routing feature such as channels or pulleys. Routing features generally do not significantly constrain translational motion of the transmission cables 62 through mechanisms such as surface friction. Sliding joints between the transmission cables 62 and the routing features can be akin to a cylindrical pair style of kinematic constraints whereby two axes of two bodies are aligned (i.e., four DoC). Moreover, the magnitude of the motions and input and other functioning at the input/handle body 50, 54 is generally equivalent to the magnitude transmitted via transmission cables 62 at the output/end effector body 52, 58. The transmission cables 62 can be semi-compliant along their axes and extents, and can be viewed as strong springs in which tension directly correlates to elongation of the transmission cables 62.

Further, in the embodiment of FIG. 5, the articulation output joint 60 can take various forms in various embodiments. The articulation output joint 60 is established between the shaft body 56 and the output/end effector body 52, 58, per this embodiment, and extends therebetween. A multitude of multi-cluster articulation joints and components can be provided at the articulation output joint 60. The articulation output joint 60 permits an axis of the output/end effector body 52, 58 to move and rotate with respect to the shaft body 56 (e.g., an end effector axis is able to rotate with respect to the shaft body 56 and with respect to the shaft axis SA). Pitch and yaw rotations, for example, can be permitted between the output/end effector body 52, 58 and the shaft body 56 via the articulation output joint 60, while other degrees of freedom therebetween can be restricted and constrained. Motions of the articulation output joint 60 are controlled by motions of the transmission cables 62. In this embodiment, the alteration and adjustment of the transmission cables tension furnished by the tensioner or tensioning mechanism/assembly and tensioning body 44 has direct impact on the control and/or effectiveness of the transmission of motions, input, and other functioning from the input/handle body 50, 54 and to the output/end effector body 52, 58.

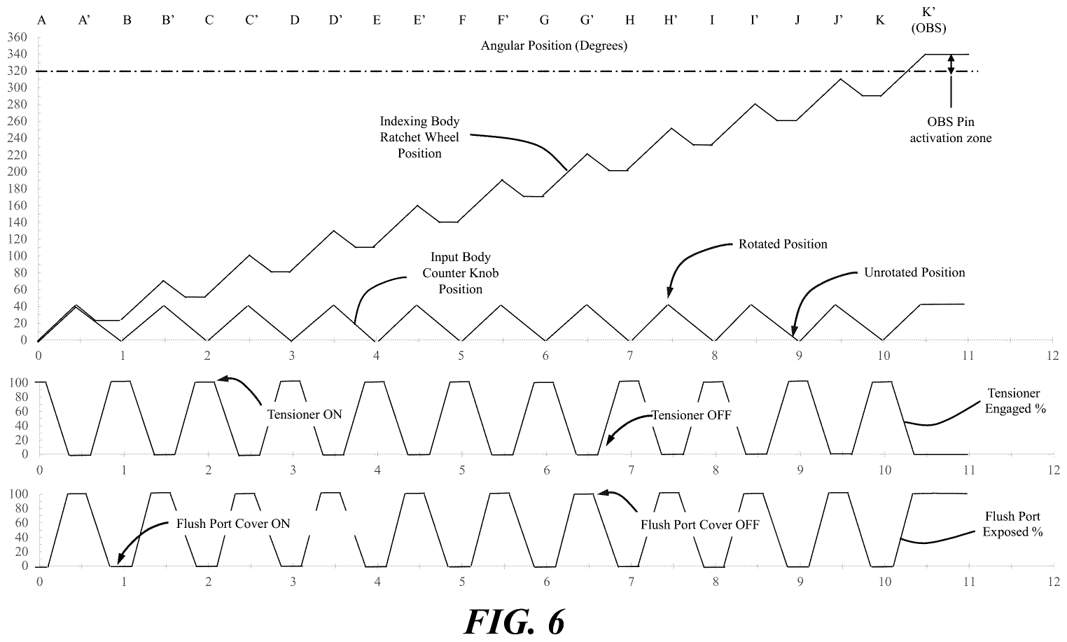

FIG. 6 presents angular positions in rotational degrees and/or states and positions according to embodiments of the cover body 42, tensioning body 44, input body 16 and counter ring body 22, and indexing body 18 and ratchet wheel body 28. The cover body 42 is denoted “Flush Port Cover” in the figure; the tensioning body 44 is denoted “Tensioner” in the figure; the input body 16 and counter ring body 22 is denoted “Input Body Counter Knob” in the figure; and the indexing body 18 and ratchet wheel body 28 is denoted “Indexing Body Ratchet Wheel” in the figure. In the use states A, B, C, D, E, F, G, H, I, J, and K, per this embodiment, the cover body 42 is in its first position and in its OFF state. In the non-use states A′, B′, C′, D′, E′, F, G′, H′, I′, J′, and K′, on the other hand, the cover body 42 is in its second position and in its ON state. In between the use and non-use states, the cover body 42 is in the midst of moving and opening or closing. Further, in the use states A, B, C, D, E, F, G, H, I, J, and K, per this embodiment, the tensioning body 44 is in its first position and in its ON state. In the non-use states A′, B′, C′, D′, E′, F, G′, H′, I′, J′, and K′, on the other hand, the tensioning body 42 is in its second position and in its OFF state. And in the use states A, B, C, D, E, F, G, H, I, J, and K, per this embodiment, the input body 16 and counter ring body 22 can be in its first position and first discrete rotational position (denoted “Unrotated Position” in the figure). In the non-use states A′, B′, C′, D′, E′, F, G′, H′, I′, J′, and K′, on the other hand, the input body 16 and counter ring body 22 can be in its second position and second discrete rotational position (denoted “Rotated Position” in the figure). Lastly, according to this embodiment, the indexing body 18 and ratchet wheel body 28 sequentially advances in a single rotational direction with respect to the first/frame body 14, 20 among the use and non-use states A, A′, B, B′, C, C′, D, D′, E, E′, F, F, G, G′, H, H′, I, I′, J, J′, K, and K′. At the non-use state K′, which is the final non-use state per this embodiment, an obsolescence triggering and activation zone (denoted “OBS Pin activation zone” in the figure) is entered and effected, whereby the obsolescence body 34 is triggered and activated.

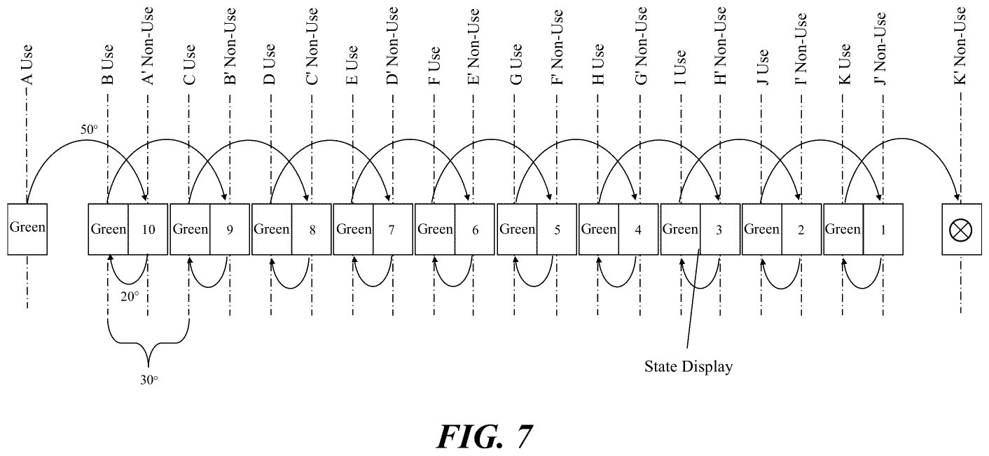

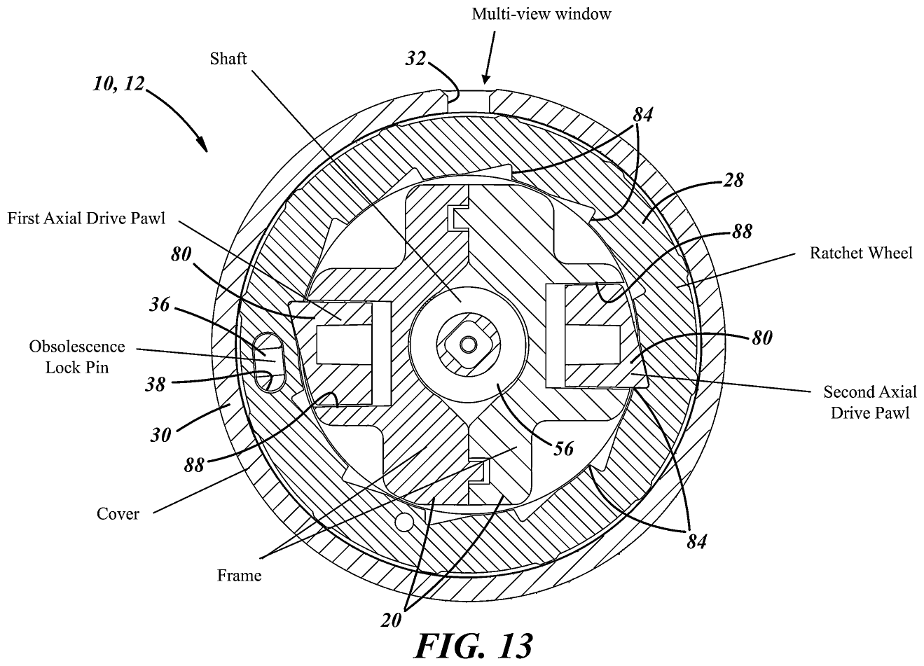

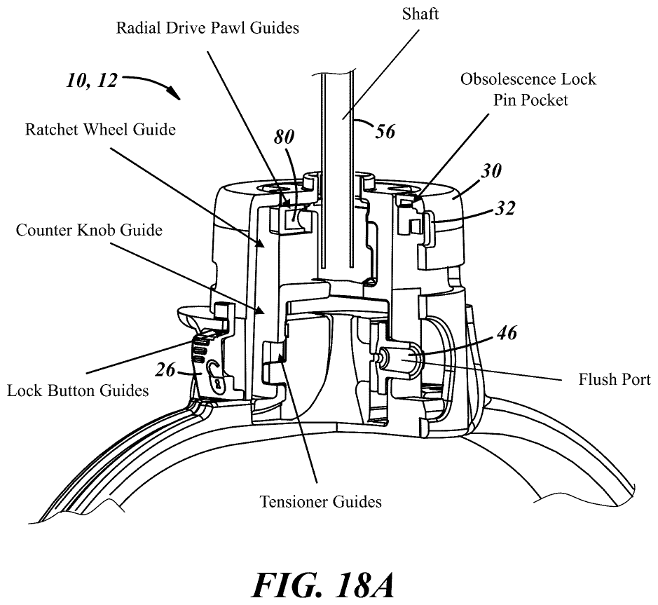

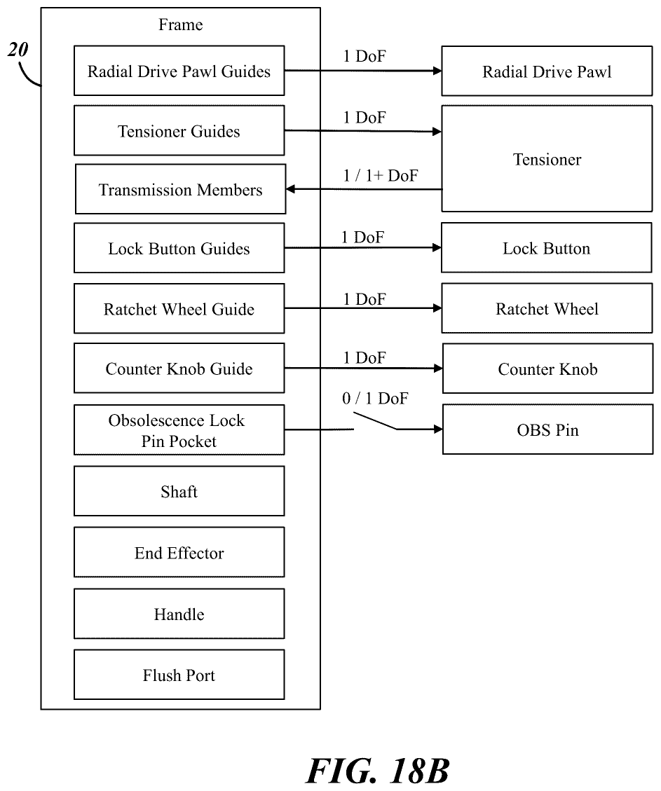

FIG. 7 presents angular and rotational advancements and retractions of the indexing body 18 and ratchet wheel body 28 as it transitions and shifts among the use and non-use states A, A′, B, B′, C, C′, D, D′, E, E′, F, F, G, G′, H, H′, I, I′, J, J′, K, and K′, according to an embodiment. Here, from the use state A to the non-use state A′, the ratchet wheel body 28 is rotated and advanced forward in the first rotational direction by approximately fifty degrees (50°). Then, to transition and shift to the use state B from the non-use state A′, the ratchet wheel body 28 is retracted rearward in the second rotational direction by approximately twenty degrees (20°). This advancement forward and retraction rearward can be effected among the remaining use and non-use states B′, C, C′, D, D′, E, E′, F, F, G, G′, H, H′, I, I′, J, J′, K, and K′, as depicted in the figure. Still, the extent and degrees of rotation can vary in other embodiments. Furthermore, FIG. 7 presents embodiments of the indicia of the ratchet wheel body 28 that is viewable at different times amid the use and non-use states of the surgical instrument assembly 10 and handheld surgical instrument 12 via the viewing window 32. The indicia can reside on a radially-outboard and exterior surface of the ratchet wheel body 28, as illustrated in FIGS. 12 and 17a. In a medical setting, a green indicia can be viewed in the use states by the user (e.g., surgeon) of the surgical instrument assembly 10 and handheld surgical instrument 12. The green indicia could be a green circle or some other representation, as examples. A numbering indicia can be viewed in the non-use states by a nurse or personnel of the central sterile processing department (CSPD). The numbering indicia can represent the number of surgical uses remaining and available of the surgical instrument assembly 10 and handheld surgical instrument 12. In the example of ten total uses, the numbering indicia can include all of the numbers and digits 1, 2, 3, 4, 5, 6, 7, 8, 9, 10. Further, according to this embodiment, a red circled X indicia can indicate the obsolescence state and the end-of-life state of the surgical instrument assembly 10 and handheld surgical instrument 12. Furthermore, in some embodiments, the ratchet wheel body 28 may consist of a display which shows the remaining uses of the surgical instrument assembly 10 and handheld surgical instrument 12 and the end-of-life status thereof. This display helps the central processing technician track the end-of-life condition of the device and make the decision to place additional orders for inventory management. The counting display can also convey safety information to the technician and cautions against a user from re-using device that is no longer safe to be reprocessed and has reached its obsolescence state. In some embodiments, the ratchet wheel body 28 display shows the number of uses the device has already experienced. This numerical change may only occur after a device has been used and reprocessed indicating to the technician the number of remaining uses. This allows users to more readily document device history and to maintain reprocessing inspection record keeping, should that be desired.

FIG. 8 presents an embodiment of the handheld surgical instrument 12 having the surgical instrument assembly 10 equipped therein. The handheld surgical instrument 12 can have other designs, constructions, components, and arrangements in other embodiments. In the embodiment of FIG. 8, the handle body 54 includes a main body 64 and a dial body 66. The main body 54 can fit within a palm of a user's hand when the user is grasping the handle body 54 in use of the handheld surgical instrument 12. The dial body 66 serves as a user input for effecting unlimited roll functionality and capabilities at the end effector body 58. The dial body 66 can rotate about a handle axis HA with respect to the main body 64. The roll functionality of the end effector body 58 is with respect to, and about, the shaft axis SA. Furthermore, intermediate bodies and joints are situated between the handle body 54 and frame body 20. The arrangement of intermediate bodies and joints furnishes the articulation input joint thereat. A handle body segment or first half ring body 68 extends from the handle body 54 and from the dial body 66. The first half ring body 68 has a fixed connection with the dial body 66. A frame body segment or second half ring body 70 extends from the frame body 20. The second half ring body 70 has a fixed connection with the frame body 20. A full ring body or deviation ring body 72 is joined to the first half ring body 68 and the second half ring body 70 via first and second joints, which can be sets of pin joints. The first joint between the first half ring body 68 and deviation ring body 72 can provide a yaw rotational degree of freedom therebetween. A first or yaw pulley 74 can be situated at one of the pin joints of the first joint and serves to capture yaw rotation of the first half ring body 68 with respect to the deviation ring body 72. The captured yaw rotation is transmitted to the end effector body 58 via yaw articulation transmission cables extending therebetween. Similarly, the second joint between the second half ring body 70 and deviation ring body 72 can provide a pitch rotational degree of freedom therebetween. A second or pitch pulley 76 can be situated at one of the pin joints of the second joint and serves to capture pitch rotation of the second half ring body 70 with respect to the deviation ring body 72. The captured pitch rotation is transmitted to the end effector body 58 via pitch articulation transmission cables extending therebetween.

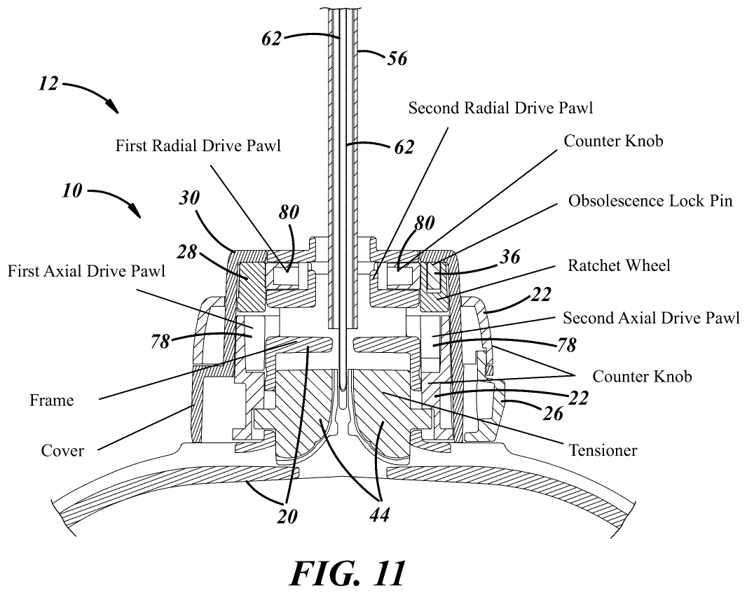

FIG. 9 shows the surgical instrument assembly 10 in its use state and condition, according to an embodiment. Here, the counter ring body 22 is in its first position and first discrete rotational position. The lock button 26 is in its ON state, and the ratchet wheel body 28 can be in one of its discrete indexed positions and discrete rotational positions. The ratchet wheel body 28 is only visible in this figure via the viewing window 32 that displays the green indicia of the ratchet wheel body 28. Further, in the use state, the tensioning body 44 can be in its first position and in its ON state, and the cover body 42 can be in its first position and in its OFF state and hence shielding the flush port 46. Moreover, in the use state, the first tension is provided to the one or more transmission cables and the flush port 46 is inaccessible. The lock body 36, if provided, remains retracted. The end effector body 58 is in its functional state. FIG. 10, in contrast, shows the surgical instrument assembly 10 in its non-use state and condition, according to an embodiment. Here, the counter ring body 22 can be in its second position and second discrete rotational position. The lock button 26 is again in its ON state (i.e., only in its OFF state to initiate transition and shifting between the use and non-use states, and vice-versa), and the ratchet wheel body 28 can be in one of its discrete indexed positions and discrete rotational positions. As before, the ratchet wheel body 28 is only visible in this figure via the viewing window 32 that displays the numbering indicia (e.g., number and digit 3) of the ratchet wheel body 28. Further, in the non-use state, the tensioning body 44 can be in its second position and in its OFF state, and the cover body 42 can be in its second position and in its ON state and hence exposing the flush port 46. Moreover, in the non-use state, the second tension is provided to the one or more transmission cables and the flush port 46 is accessible. The end effector body 52, 58 is in its non-functional state.

With general reference now to FIGS. 11, 13, and 14, sectional views of the surgical instrument assembly 10 are presented for demonstrative purposes. Here, it is evident that the counter ring body 22 has both an external body portion and an internal body portion; the external body portion and internal body portions have a fixed connection with each other and are fully constrained with respect to each other; in this regard, from a kinematics perspective, the external and internal body portions can constitute a single body. Furthermore, per an embodiment, a ratcheting functionality and capability can be furnished with rotational movements of the ratchet wheel body 28. The ratcheting functionality can have various designs, constructions, and components in various embodiments. In the embodiment of the figures, the ratcheting functionality is carried out via a first set of drive pawls 78, a second set of drive pawls 80, first drive pawl teeth 82, and second drive pawl teeth 84; still, in other embodiments there could be more or less components including a single drive pawl and single drive pawl teeth, and the drive pawl and drive pawl teeth could have other locations than described herein. The first set of drive pawls 78, second set of drive pawls 80, first drive pawl teeth 82, and second drive pawl teeth 84 can be components of the use count mechanism/assembly.

The ratcheting functionality and its components serve to furnish rotational drive and movement of the ratchet wheel body 28 in an intended rotational direction (e.g., clockwise or counterclockwise), and precludes and prevents unwanted rotational drive and movement of the ratchet wheel body 28 in the opposite rotational direction (e.g., the other counterclockwise or clockwise). Rotational drive and concomitant incrementation of the use count and corresponding indicia of the ratchet wheel body 28 and of the surgical instrument assembly 10 hence only occurs in the intended rotational direction, and does not occur in the opposite, unintended rotational direction. Irreversibility of the use count mechanism/assembly is thereby furnished, precluding a misuse scenario in which incrementation of the use count would occur in the opposite, unintended rotational direction and inaccurate representations and information would consequently be displayed and conveyed to the user. The counter ring body 22 can still be rotated in the opposite rotational direction, per this embodiment, such as for the purpose of bringing the tensioning body 44 to its ON state.

In FIGS. 11, 13, and 14, the first set of drive pawls 78 is an axial set of drive pawls that are directed in the axial direction and axially upward. The first set of drive pawls 78 engage with the first drive pawl teeth 82 via teeth-to-teeth engagement and abutment. The engagement is in the axial direction, per this embodiment. There are a pair of the first set of drive pawls 78 in this embodiment: a first drive pawl located and positioned on one side of the counter ring body 22 with respect to the ratchet wheel body 28, and a second drive pawl located and positioned on the opposite side of the counter ring body 22 with respect to the ratchet wheel body 28. The first and second drive pawls can be one-hundred-and-eighty rotational degrees (180°) apart from each other. The first set of drive pawls 78 is carried by the counter ring body 22 according to this embodiment. Each of the first and second drive pawls of the first set of drive pawls 78 is seated within cavities 86 residing and defined in the counter ring body 22. The cavities 86 serve as guides for movements of the first and second drive pawls of the first set of drive pawls 78. A single DoF is provided between the first set of drive pawls 78 and the counter ring body 22. The single DoF can be a single translational DoF, per an embodiment. A prismatic joint can be established between first set of drive pawls 78 and the counter ring body 22. A spring or other biasing member or compliant connector urges and biases the first and second drive pawls of the first set of drive pawls 78 axially outboard and upward and toward the ratchet wheel body 28. Distal ends of the first and second drive pawls of the first set of drive pawls 78 are inclined and slanted for suitable ratcheting engagement with the first drive pawl teeth 82. The distal ends can exhibit a shape and size that complements that of the first drive pawl teeth 82. The ratchet wheel body 28 and counter ring body 22 are mechanically coupled together via the first set of drive pawls 78 and first drive pawl teeth 82.

Furthermore, the second set of drive pawls 80 is a radial set of drive pawls that are directed in the radial direction and radially outboard; in this regard, the first and second sets of drive pawls 78, 80 have an orthogonal arrangement relative to each other. The second set of drive pawls 80 engage with the second drive pawl teeth 84 via teeth-to-teeth engagement and abutment. The engagement is in the radial direction, per this embodiment. There are a pair of the second set of drive pawls 80 in this embodiment: a first drive pawl located and positioned on one side of the frame body 20 with respect to the ratchet wheel body 28, and a second drive pawl located and positioned on the opposite side of the frame body 20 with respect to the ratchet wheel body 28. The first and second drive pawls can be one-hundred-and-eighty rotational degrees (180°) apart from each other. The second set of drive pawls 80 is carried by the frame body 20 according to this embodiment. Each of the first and second drive pawls of the second set of drive pawls 80 is seated within cavities 88 residing and defined in the frame body 20. The cavities 88 serve as guides for movements of the first and second drive pawls of the second set of drive pawls 80. A single DoF is provided between the second set of drive pawls 80 and the frame body 20. The single DoF can be a single translational DoF, per an embodiment. A prismatic joint can be established between second set of drive pawls 80 and the frame body 20. A spring or other biasing member or compliant connector urges and biases the first and second drive pawls of the second set of drive pawls 80 radially outboard and toward the ratchet wheel body 28. Distal ends of the first and second drive pawls of the second set of drive pawls 80 are inclined and slanted for suitable ratcheting engagement with the second drive pawl teeth 84. The distal ends can exhibit a shape and size that complements that of the second drive pawl teeth 84. The ratchet wheel body 28 and frame body 20 are mechanically coupled together via the second set of drive pawls 80 and second drive pawl teeth 84.

Further, and with reference now to FIG. 17a, the first and second drive pawl teeth 82, 84 are located at, and carried by, the ratchet wheel body 28 according to this embodiment. Here, the first and second drive pawl teeth 82, 84 are integral with, and unitary extensions of, the ratchet wheel body 28. The first and second drive pawl teeth 82, 84 each include a multitude of individual teeth that extend wholly around the circumference of the ratchet wheel body 28. The first drive pawl teeth 82 are axial drive pawl teeth that are directed in the axial direction and axially downward. The first drive pawl teeth 82 project from an axially-bottom surface of the ratchet wheel body 28. The second drive pawl teeth 84, on the other hand, are radial drive pawl teeth that are directed in the radial direction and radially inward; in this regard, the first and second drive pawl teeth 82, 84 have an orthogonal arrangement relative to each other. The second drive pawl teeth 84 project from a radially-inboard surface of the ratchet wheel body 28. Furthermore, having the axial and radial drive pawl teeth at the ratchet wheel body 28 can establish a flattened and co-called dwell zone amid rotation, per an embodiment. This produces a “partial back drive” mechanism, in which the ratchet wheel body 28 partially reverses rotational position by a pre-determined amount that is less than one full rotation, until the axial or radial drive pawl teeth (depending on embodiment) engage and stops the reverse rotation. This partial back-driven functionality allows a secondary set of display symbols that may only be visible in the reverse direction user input. This process allows a dedicated set of symbols and indicia of the ratchet wheel body 28 to be visible to one set of users, and another set of symbols and indicia to be visible to another. For example, a set of ready-to-use state symbol and indicia may be displayed to only surgeons when they rotate the counter ring body 22 clockwise as a tensioning and surgical usage procedure, and a set of “uses remaining” symbol and indicia may be displayed to only reprocessing technicians when they rotate the counter ring body 22 counterclockwise as a de-tensioning and instrument reprocessing procedure. Moreover, enabling the ratchet wheel body 28 to be partially back-driven allows for the movement of the counter ring body 22 to be unique relative to the movement of the ratchet wheel body 28. The counter ring body 22 is no longer limited based on the intended use count of the rachet wheel body 28. For example, if the counter ring body 22 rotates 50°, and if the ratchet wheel body 28 was unable to be partially back-driven and rotate back 20°, then the ratchet wheel body 28 would be limited, since it is a rotational mechanism to only seven use states in total. Considering the ability of the ratchet wheel body 28 to be partially back-drivable and rotate back 20° the ratchet wheel body 28 is now able to comprise of twelve use states in total, per an embodiment. Furthermore, there are system benefits to having the input to the counter ring body 22 be a large value. The counter ring body 22 acts upon the tensioner body 44 as well as exposes the flush port 46, it is advantageous per an embodiment for the counter ring body 22 to comprise of a large displacement as it allows the system to achieve a greater variation in the use state compared to the non-use state. Still, in alternative embodiments, the ratchet wheel body 28 could be a single body within a multi-body rotary gear display in which the use count mechanism/assembly is able to index similar to a standard tally counter which there are multiple wheels with each wheel used to displace a single digit.

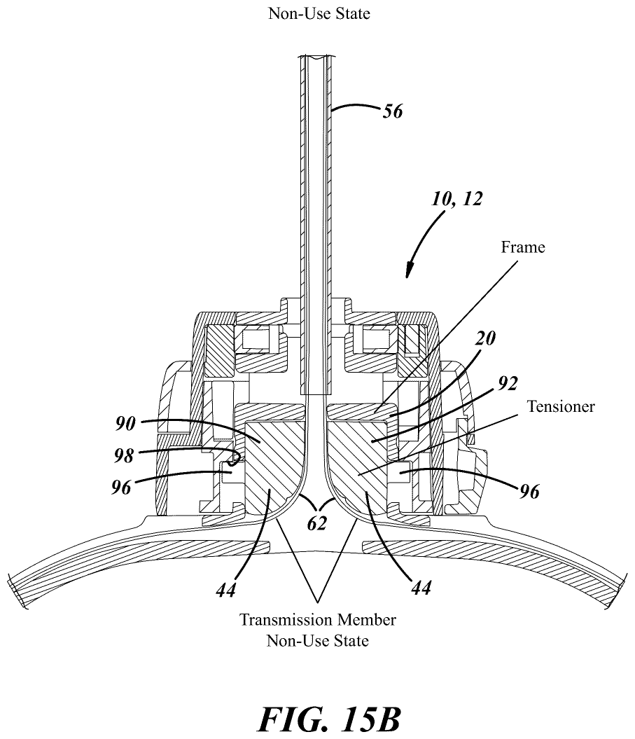

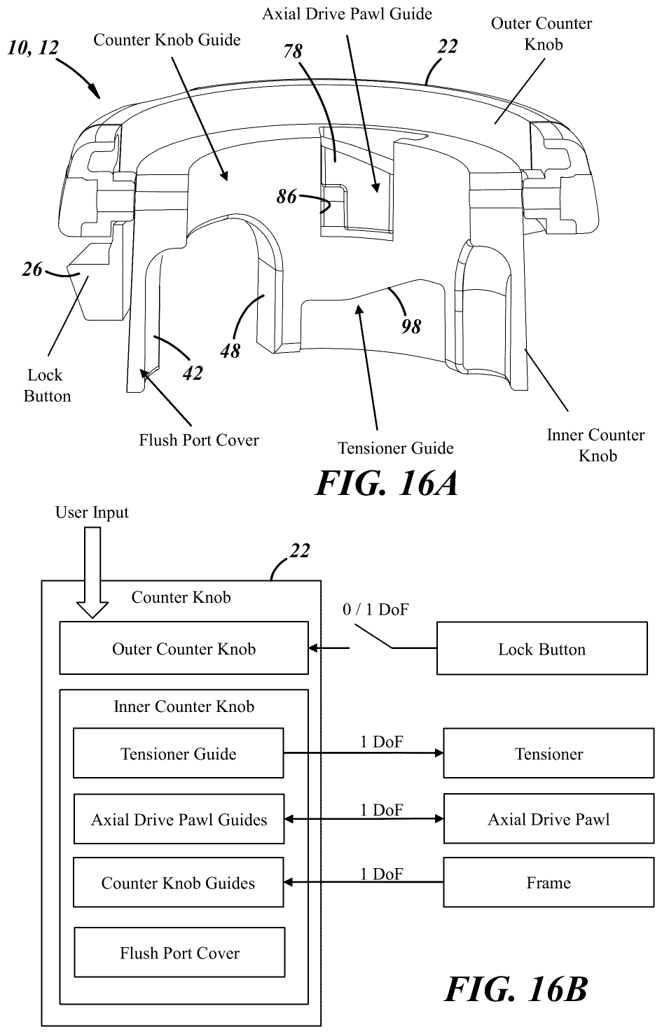

With reference now to FIGS. 15a, 15b, and 15c, the tensioner or tensioning mechanism/assembly includes the tensioning body 44. The tensioner or tensioning mechanism/assembly can have various designs, constructions, and components in various embodiments. In the embodiment of the figures, the tensioning body 44 can have a first tensioning body portion 90 and a second tensioning body portion 92. The first tensioning body portion 90 can interact with one or more of the transmission cables 62, and the second tensioning body portion 92 can interact with another one or more of the transmission cables 62. Working surfaces 94 of the tensioning body 44 and first and second tensioning body portions 90, 92 can engage and guide a section of the transmission cables 62 that spans therealong. A tensioning cam or peg 96 projects radially outboard from the tensioning body 44, per this embodiment. The tensioning peg 96 rides in a cam guide and profile surface 98 residing and defined in the counter ring body 22, as depicted in FIGS. 14 and 16a. A cam-follower relationship and arrangement is established therebetween. Inter-engagement between the tensioning peg 96 and cam profile surface 98 can provide a rotational DoF therebetween and a translational DoF therebetween, and serves to drive actuation of the tensioning body 44. The cam profile surface 98 is designed whereby a one DoF screw pair can be provided that furnishes force and motion to the tensioning body 44 when the counter ring body 22 rotates with respect to the frame body 20. For instance, in one rotational direction movement of the counter ring body 22 relative to the frame body 20, the tensioning body 44 translates in a first direction; and, conversely, in an opposite rotational direction movement of the counter ring body 22 relative to the frame body 20, the tensioning body 44 translates in a second, opposite direction. The tensioning body 44 itself does not rotate when the counter ring body 22 rotates, per this embodiment, and rather remains static relative thereto.

A spring or other biasing member or body or compliant connector urges and biases the tensioning body 44 axially upward with respect to the counter ring body 22, urging and biasing surface-to-surface engagement between the tensioning peg 96 and cam profile surface 98. The tensioning body 44 moves and translates axially with respect to the counter ring body 22 and with respect to the frame body 20 when the surgical instrument assembly 10 and handheld surgical instrument 12 are in the use and non-use states. The axial movement is relative to the shaft axis SA. In FIG. 15a, in the use state, the tensioning body 44 has a first position or first axial position relative to the counter ring body 22 and the frame body 20. A first transmission cable path length is provided when the tensioning body 44 is in its first axial position. The first tension is provided to the transmission cables 62 via the first transmission cable path length and first axial position. The transmission cables 62 are engaged taut against the working surfaces 94, per this embodiment, when the first transmission cable path length is provided and when the tensioning body 44 is in its first axial position. The first axial position is established when the tensioning peg 96 is positioned at an axially-lower section of the cam profile surface 98. Further, in FIG. 15b in the non-use state, on the other hand, the tensioning body 44 has a second position or second axial position relative to the counter ring body 22 and the frame body 20. A second transmission cable path length is provided when the tensioning body 44 is in its second axial position. The first transmission cable path length is greater in value than that of the second transmission cable path length, according to this embodiment. The second tension is provided to the transmission cables 62 via the second transmission cable path length and second axial position. The transmission cables 62 can be engaged against the working surfaces 94, per this embodiment, when the second transmission cable path length is provided and when the tensioning body 44 is in its second axial position. Engagement here can be less taut than that of the first transmission cable path length. The second axial position is established when the tensioning peg 96 is positioned at an axially-upper section of the cam profile surface 98.

Further, according to an embodiment, in a rotational position, the counter ring body 22 provides full axial constraint between the tensioning body 44 and the frame body 20. In this position, a transmission member guide path of the tensioning body 44 is directly constrained by the frame body 20 through the counter ring body 22 and allows efficient transmission of forces or signal through the tensioning body 44. In another rotational position of the counter ring body 22 (alternative state of the counting mechanism), the tensioning body 44 is no longer rigidly axially constrained between the frame body 20 and the counter ring body 22. When the tensioning body 44 travels axially, the transmission member guide path translates, resulting in the overall cable path of the device to lengthen and reduces the transmission member tension. In this state of the device, the tensioning body 44 may also be biased axially to one direction with a force that is determined by a compliance member (e.g., a spring). The combination of these effects allows the tensioning body 44 to travel axially to balance the forces in the system in response to the transmission member changing tension during use or thermal cycling. During thermal cycling, a system without this tensioner limiter (tension relief) would result in the tension in the system increasing as the transmission member path will lengthen with respect to the transmission member length due to differences in coefficients of thermal expansion. The tensioning body 44 reduces the sensitivity of the transmission member path effect on the tension of the system as the transmission member path is more compliant when the tensioning body 44 is active which means it can compensate for variation in path length without changing the transmission member length. The tensioning body 44 not only provides tension relief to the system but also can be utilized as a mechanism which establishes a minimum tension in the system. This is beneficial as a zero tension may be undesired in some configurations as the device may be able to come apart or the transmission members will come out of the transmission member path if the system has zero tension. Furthermore, once reprocessed, per an embodiment and application, a user can activate the counter ring body 22 in the reverse direction to re-tension the device for use.

Moreover, according to an embodiment, the minimum tension in the transmission cables 62 is greater than zero when the surgical instrument assembly 10 and handheld surgical instrument 12 is in the use state. Further, the position of the tensioning body 44 is constrained by the position of the cam profile surface 98 and frame body 20. The interface between the transmission cables 62 and the transmission member guide path located on the tensioning body 44 may be a cylindrical joint with 2 DoF allowing for rotation and axial movement per unit length of the transmission cables 62. More generally due to geometry of common the interface between these two bodies may be at minimum a prismatic joint with 1 DoF allowing axial motion per unit length of the transmission cables 62. Further, per an embodiment, the cam profile surface 98 can be unidirectional whereby it would limit the position of the tensioning body 44 in one axial direction by not the other. In an embodiment in which the cam function serves as a one DoF screw pair, the tensioning body 44 may be permitted to translate within tensioner slot guides in a range that is between a frame body hard stop and the cam profile surface 98. Further, when in the non-use state, the position of the tensioning body 44 can be directly proportional to the compliant feature or connecting body of the tensioning body 44, when provided, such as a spring. As the position of the tensioning body 44 is no longer simultaneously constrained by the rigid body of the frame body 20 and the cam profile surface 98 of the counter ring body 22, instead the tensioning body 44 has freedom to translate within the frame body 20.

Further, in an alternative embodiment, the tensioning body 44 need not have a spring or other biasing member or body or compliant connector. Here, as the surgical instrument assembly 10 and handheld surgical instrument 12 shift from the use state to the non-use state, the tensioning body 44 can, in a sense, float freely within a tensioner slot guide resulting in zero tension provided to the transmission cables 62. Absent any biasing exertion, all DoC between the transmission cables 62 and the tensioning body 44 is removed. Tension provided to the transmission cables 62 can be maintained at zero so long as suitable clearance is provided in the system.

Furthermore, an embodiment providing thermal counting may be provided. Here, the counting mechanism may consist of smart actuators such as shape memory alloy (SMA). An example of an SMA is a thermally activated bi-metallic strip or a nitinol wire. These SMAs can generate relatively large displacements when activated, due to the geometry and construction of the SMA being determined by the amount of displacement generated per unit change in temperature. Variations of the SMA include bi-stable mechanism which toggle when a target temperature is reached, or a variable configuration, in which each incremental change in temperature results in an incremental change in shape. The ability of an SMA to restore back to its nominal position after experiencing thermal deformation allows the material to act as an actuation mechanism. This mechanism allows multiple cycles to be performed and used as an integral to an indexing mechanism which can count various events. The use of thermally activated materials such as these can provide benefits to the general counting mechanism.

An example, of how thermally activated materials can be beneficial to previously described mechanisms, would be used to generate the input force and generate displacement of the counter ring body 22. This prevents the necessity for external user input to cycle the surgical instrument assembly 10. A body can be introduced into the system which comprises of a SMA material that deforms in response to temperature change. The absence of user input both eliminates a user action as well as automates the counting mechanism, thus enabling the counting mechanism to self-index and count in absence of user presence. This may be beneficial in the application of a surgical device in which reprocessing is completed through a method of steam sterilization. During a steam sterilization cycle the temperature increases to ˜135 degree C. before restoring back to ambient of ˜23 degrees C.; this change in temperature is suitable to cause actuation based on the construction and geometry of a SMA. Within the steam sterilization cycle the device may be placed in a tray or wrapped in a barrier to ensure sterility when the cycle is complete, therefore the device may be unreachable by user interaction for the time which it is wrapped. Prior to unwrapping, the deformation of the SMA at elevated temperatures would provide the necessary input force and displacement to advance the counting mechanism and when ambient temperatures are restored the SMA will generate force and displacement in the opposite direction, thus restoring the counting mechanism to the initial state but index one forward.

Alternative to being the input force of the counting mechanism, an SMA has other practical applications within the counting mechanism. An SMA can replace any of the compliant bodies or springs within the system. Having a spring which is only active at set temperature ranges may allow for the counting mechanism to only be activated at target temperatures. For example, using an SMA as a member within the lock button 26 prevents the user from rotating the counter ring body 22 unless temperature of the device is below (or above) a certain value. In practice, the thermally actuated material configuration is an automatic safety switch to the system. This occurs by the material changing the present state of the counting mechanism at an elevated temperature. The thermally activated material can assist in preventing permanent damage to the device due to the stress build up caused by the thermal deformation of dissimilar materials.

Further, in some embodiments the counting mechanism can act in reverse, rather than a user input driving the counting mechanism and de-tensioning the device. The counting mechanism is driven by the tensioning body 44 such that when the tensioning body 44 experiences a predetermined maximum force it drives the ratchet wheel body 28 forward and indexes the system. In this embodiment, the counter counts the number of over-stress events of the device. This can be useful as over-stress can lead to accelerated device wear or even device damage, the tracked information can determine when the device would need to be obsoleted. Further, in alternate embodiments, the counter is engaged with an electrical port, and the insertion of an electrical connector to the electrical port indexes the ratchet wheel body 28. In some embodiments, the counter input body mechanism interfaces with a sterilization tray. The insertion of the device onto the sterilization tray indexes the ratchet wheel body 28. In some embodiments, a specialized tool may need to be inserted to increment the counter; here, the tool will only be provided to trained sterile processing personnel.

Further, per various embodiments, the rotation of the counter ring body 22 causes rotation of the ratchet wheel body 28. The angle per incrementation on the ratchet wheel body 28 can vary on the number of uses built into the device counter, but the angle per rotation for the counter ring body 22 may be a set value that is needed to generate enough rotation to fully expose the flush port 46 or enough travel to fully de-tension the transmission cables 62. In some embodiments, a dwell is built into the rotation engagements such that some components in the counter assembly may finish rotation first but continues to rotate to allow other components to finish rotation.

In another embodiment, the tensioner or tensioning mechanism/assembly can involve a pulley type body which has functionality as a transmission member path which is in mechanical engagement with the counter ring body 22 and the transmission cables 62. The activation of the counter ring body 22 subsequently displaces the pulley body in such a way that the transmission member path length increases and the resulting transmission member tension decreases. In some embodiments, several of these bodies may be arranged in a staggered configuration to achieve different tension reduction. In some embodiments, the angular displacement of the pulley bodies may be modulated and secured with rack and pawl features. In some embodiments, the angular displacement of the pulley bodies may be mechanically engaged with the frame body 20 with a compliant connector to maintain the same transmission member tension in response to thermal expansion stress.

An embodiment has an effect on different keying features within the assembly. An example of obsolescence of the counter affecting other features includes the tensioner or tensioning mechanism/assembly of the surgical instrument assembly 10. The tensioner or tensioning mechanism/assembly has a functional effect based on the state of the counter. During a use state, the tensioning body 44 is fully constrained between the frame body 20 and counter ring body 22. This directly correlates with the compression spring sitting in a non-fired state covering the flush port 46. During a non-use state, the compression spring is fired with the tensioning peg 96 changing orientation and allowing flush port accessibility. It is during the obsolescence state that the drive pawls and obsolescence pin is fired to cancel the tensioning mechanism within the device. In correlation with the prior feature, the flush port 46 is also no longer activated when the device reaches the obsolescence state. When the counter reaches the obsolescence state, the flush port cover body 42 of the counter ring body 22 blocks the flush port 46 and restricts the device from having any fluid inserted within the device.

Further, in other embodiments, a main body such as the frame body 20 contains a lock-out pawl that is mechanically engaged with the main body of the device and the ratchet wheel body 28, which contains a lock-out groove. The overall assembly has two states. In one state, the lock out pawl enforces a one-way incrementation of the counter index dial through a ratchet engagement feature. In another state, the lock-out pawl extends radially into a groove on the ratchet wheel body 28, directly and permanently preventing rotation of the counter index dial in both directions and indirectly preventing the rotation of the cam collar in both directions. This prevents the re-tensioning of the device. In some embodiments, the lock-out state groove is above the “zero” or non-usable count position of the counter index dial. In some embodiments, an axial lock-out pawl on the ratchet wheel body 28 may also extend into a groove on a static component of the device to achieve lock-out.

Further, in some embodiments, a severing component may be deployed by a feature on the counter ring body 22. When the ratchet wheel body 28 rotates into a pre-determined location, a lock pawl may extend into the severing component and dislodge the safety pin to cause the device cables to be severed. In some embodiments, the electrical connection cables may be severed instead.

Further, in some embodiments, a shield may be deployed by a feature on the ratchet wheel body 28. When the ratchet wheel body 28 rotates onto a pre-determined location, a shield may be deployed to obstruct the electrical connection port permanently.

A fully accessible counting mechanism designed to accurately track the number of times a medical device has been sterile processed is provided. The mechanism, per varying embodiments, utilizes a combination of mechanical numerical indexing to display remaining use count, a lock-out system to prevent unsafe usage past the pre-determined use count, a back-drive rotation lock-out to prevent reversing count, a rotational cover to hide cleaning ports and ensure sterility during surgical use, and an activation of a cable de-tensioning system to relief thermal expansion stress during steam sterilization.

Further, in various embodiments, input to the counter ring body 22 may no longer be external user input; rather, the counter ring body 22 could be activated via an internal SMA such as a bi-metallic strip or nitinol wire. The applied load of the SMA may be able to change the state of the device from the use state to the non-use state, but applying a force in a single direction while heated and then applying a restorative force when cooled. Alternative, an SMA may be used to change the state of the system in a single direction by applying a load resulting in unidirectional motion when thermally cycled. This could for example change the state from the non-use state to the use state, but not from the use state to the non-use state. Further, in various embodiments, one or more interlocks within the surgical instrument assembly 10 could be thermally-activated, as described; this could involve the cover body 42 self-closing without impact to other mechanisms/assemblies. Further, in various embodiments, there may be a seal within the device which when active prevents bioburden or contaminates from entering freely while in the use state, but the seal can be deactivated during reprocessing. A SMA could deactivate the seal or other interlock during thermal cycling enabling the device to be reprocessed in a different state than the use state.

The figures are not necessarily to scale, and some features may be exaggerated or minimized to show key details of components. Therefore, specific structural and functional details disclosed herein are not to be interpreted as limiting, but merely as a representative basis for teaching one skilled in the art to variously employ the present invention.

This disclosure is not intended to limit the scope to medical devices or the healthcare industry. For example, the designs and constructions and components and assemblies described herein may be applied to conventional electro-mechanical control systems and industrial applications.

Furthermore, in general, while a multitude of embodiments have been depicted and described with a multitude of components in each embodiment, in alternative embodiments of the surgical instrument assembly the components of various embodiments could be intermixed, combined, and/or exchanged for one another. In other words, components described in connection with a particular embodiment are not necessarily exclusive to that particular embodiment.

As used herein, the terms “general” and “generally” and “substantially” are intended to account for the inherent degree of variance and imprecision that is often attributed to, and often accompanies, any design and manufacturing process, including engineering tolerances—and without deviation from the relevant functionality and intended outcome—such that mathematical precision and exactitude is not implied and, in some instances, is not possible. In other instances, the terms “general” and “generally” and “substantially” are intended to represent the inherent degree of uncertainty that is often attributed to any quantitative comparison, value, and measurement calculation, or other representation.

It is to be understood that the foregoing is a description of one or more aspects of the disclosure. The disclosure is not limited to the particular embodiment(s) disclosed herein, but rather is defined solely by the claims below. Furthermore, the statements contained in the foregoing description relate to particular embodiments and are not to be construed as limitations on the scope of the disclosure or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A surgical instrument assembly, comprising:
a frame body;
an input body moveable with respect to said frame body and having a first position with respect to said frame body and a second position with respect to said frame body; and
an indexing body prompted to move with respect to said frame body via said input body, said indexing body moveable to a plurality of indexed positions with respect to said frame body;
wherein, upon attainment by said indexing body of a final indexed position of said plurality of indexed positions, movement of said input body with respect to said frame body is fully constrained.

2. The surgical instrument assembly as set forth in claim 1, wherein said indexing body advances to each of said plurality of indexed positions via a forward direction and is precluded from movement in an opposite rearward direction.

3. The surgical instrument assembly as set forth in claim 1, further comprising a lock body and, upon attainment by said indexing body of the final indexed position, movement of said input body with respect to said frame body is fully constrained via said lock body.

4. The surgical instrument assembly as set forth in claim 1, further comprising a lock body and, upon attainment by said indexing body of the final indexed position, movement of said indexing body with respect to said frame body is fully constrained via said lock body.

5. The surgical instrument assembly as set forth in claim 3, wherein said lock body is prompted to fully constrain movement of said input body with respect to said frame body only upon attainment by said indexing body of the final indexed position and when said lock body and a recess are brought into alignment with each other.

6. The surgical instrument assembly as set forth in claim 4, wherein said lock body is prompted to fully constrain movement of said indexing body with respect to said frame body only upon attainment by said indexing body of the final indexed position and when said lock body and a recess are brought into alignment with each other.

7. The surgical instrument assembly as set forth in claim 1, wherein said input body is a counter ring body, said counter ring body moveable with respect to said frame body via a rotational degree of freedom and having a first discrete rotational position and a second discrete rotational position.

8. The surgical instrument assembly as set forth in claim 7, wherein the rotational degree of freedom between said counter ring body and said frame body constitutes a sole degree of freedom therebetween.

9. The surgical instrument assembly as set forth in claim 1, further comprising at least one tensioning body prompted to move with respect to said frame body via said input body, wherein, when said input body is in the first position, a first tension is provided to at least one transmission cable via said at least one tensioning body, and wherein movement of said input body from the first position and to the second position moves said at least one tensioning body with respect to said frame body and a second tension is provided to the at least one transmission cable via said at least one tensioning body, the second tension being less than the first tension.

10. The surgical instrument assembly as set forth in claim 9, further comprising a cover body prompted to move with respect to said frame body via said input body, wherein, when said input body is in the first position, said cover body renders a flush port inaccessible, and wherein, movement of said input body from the first position and to the second position moves said cover body and renders the flush port accessible.

11. The surgical instrument assembly as set forth in claim 10, wherein each of said plurality of indexed positions constitutes a use count of the surgical instrument assembly, and advancement of said indexing body to each of said plurality of indexed positions increments the use count of the surgical instrument assembly.

12. The surgical instrument assembly as set forth in claim 11, wherein, a single input action to said input body moves said input body from the first position and to the second position and concurrently incites: i) advancement of a use count increment of the surgical instrument assembly, ii) provision of the second tension to the at least one transmission cable, and iii) accessibility of the flush port.

13. The surgical instrument assembly as set forth in claim 12, wherein a second single input action to said input body moves said input body from the second position and to the first position and concurrently incites: i) provision of the first tension to the at least one transmission cable, and ii) inaccessibility of the flush port.

14. The surgical instrument assembly as set forth in claim 9, further comprising a cam body, said cam body having a connection with said input body, said cam body moving with said input body relative to said frame body, said cam body interacting with said indexing body and with said tensioning body and inciting movement of said indexing body and of said tensioning body.

15. The surgical instrument assembly as set forth in claim 1, further comprising a cover body prompted to move with respect to said frame body via said input body, wherein, when said input body is in the first position, said cover body renders a flush port inaccessible, and wherein, movement of said input body from the first position and to the second position moves said cover body and the flush port is rendered accessible.

16. The surgical instrument assembly as set forth in claim 1, wherein said input body is a counter ring body and said indexing body is a ratchet wheel body.

17. The surgical instrument assembly as set forth in claim 16, wherein said counter ring body is moved with respect to said frame body via user input action.

18. The surgical instrument assembly as set forth in claim 1, further comprising a temperature-deformable body that, upon experiencing an increase in temperature, prompts movement of said input body with respect to said frame body.

19. The surgical instrument assembly as set forth in claim 1, further comprising a temperature-deformable body that, upon experiencing an increase in temperature, permits movement of said input body with respect to said frame body, and wherein movement of said input body with respect to said frame body is constrained prior to being permitted via said temperature-deformable body.

20. The surgical instrument assembly as set forth in claim 1, wherein each of said plurality of indexed positions constitutes a use count of the surgical instrument assembly, advancement of said indexing body to each of said plurality of indexed positions increments the use count of the surgical instrument assembly, and a final indexed position of said plurality of indexed positions being a final use count of the surgical instrument assembly.

21. The surgical instrument assembly as set forth in claim 20, wherein the final use count is resettable for subsequent use of the surgical instrument assembly.

22. The surgical instrument assembly as set forth in claim 20, wherein, when said input body is in the second position, a use count indicia is displayed representative of the use count of the surgical instrument assembly.

23. The surgical instrument assembly as set forth in claim 1, wherein, when said input body is in the first position, a use state of the surgical instrument assembly is established, and when said input body is in the second position, a non-use state of the surgical instrument assembly is established.

24. The surgical instrument assembly as set forth in claim 23, further comprising a retention body, said retention body maintaining said input body in the first position and in the second position and, upon actuation of said retention body, said input body is moveable from the first position and from the second position.

25. The surgical instrument assembly as set forth in claim 23, wherein, when the surgical instrument assembly is in the non-use state, the surgical instrument assembly is in a reprocessing-ready state.

26. The surgical instrument assembly as set forth in claim 23, further comprising a cover body prompted to move with respect to said frame body via said input body, wherein, when the surgical instrument assembly is in the use state, said cover body renders a flush port inaccessible, and wherein, when the surgical instrument assembly is in the non-use state, said cover body renders the flush port accessible.

27. A surgical instrument assembly, comprising:
a frame body;
an input body moveable with respect to said frame body and having a first position with respect to said frame body and a second position with respect to said frame body; and
a tensioning body prompted to move with respect to said frame body via said input body;
wherein the surgical instrument assembly has a use state and a non-use state, the use state establishable when said input body is in the first position, the non-use state establishable when said input body is in the second position; and
wherein, when the surgical instrument assembly is in the use state, a first tension is provided to at least one transmission cable via said tensioning body, and wherein, movement of said input body from the first position and to the second position provides a second tension to the at least one transmission cable via said tensioning body, the second tension being less than the first tension.

28. The surgical instrument assembly as set forth in claim 27, wherein a first transmission cable path length is provided via said tensioning body when said input body is in the first position, and a second transmission cable path length is provided via said tensioning body when said input body is in the second position, the first transmission cable path length being greater than the second transmission cable path length.

29. The surgical instrument assembly as set forth in claim 27, wherein said tensioning body moves in a first axial direction with respect to said frame body when said input body moves from the first position and to the second position, and said tensioning body moves in an opposite second axial direction with respect to said frame body when said input body moves from the second position and to the first position.

30. The surgical instrument assembly as set forth in claim 27, further comprising a biasing body biasing said tensioning body to impart the first tension.

31. The surgical instrument assembly as set forth in claim 27, further comprising a retention body, said retention body maintaining said input body in the first position and in the second position and, upon actuation of said retention body, said input body is moveable from the first position and from the second position.

32. The surgical instrument assembly as set forth in claim 27, further comprising a lock body and a recess, wherein, upon attainment of a final use state of the surgical instrument assembly, said lock body is received in said recess and said input body is constrained from movement to the first position, establishment of the use state is hence precluded and provision of the first tension to the at least one transmission cable is hence prevented.

33. The surgical instrument assembly as set forth in claim 27, further comprising an indexing body moveable to a plurality of indexed positions, each of said plurality of indexed positions constitutes a use count of the surgical instrument assembly, and each establishment of the use state advances said indexing body to one of said plurality of indexed positions and increments the use count of the surgical instrument assembly.

34. The surgical instrument assembly as set forth in claim 27, further comprising a cover body moveable with respect to said frame body, wherein, when the surgical instrument assembly is in the use state, said cover body renders a flush port of the surgical instrument assembly inaccessible, and, when the surgical instrument assembly is in the non-use state, movement of said cover body renders the flush port accessible.

35. A surgical instrument assembly, comprising:
a frame body;
an input body moveable with respect to said frame body; and
an indexing body prompted to move with respect to said frame body via said input body, said indexing body moveable to a plurality of indexed positions with respect to said frame body, wherein each of said plurality of indexed positions constitutes a use count of the surgical instrument assembly, advancement of said indexing body to each of said plurality of indexed positions increments the use count of the surgical instrument assembly.

36. The surgical instrument assembly as set forth in claim 35, further comprising a tensioning body moveable with respect to said frame body, wherein, when said input body is in a first position with respect to said frame body, a first tension is provided to at least one transmission cable via said tensioning body, and, when said input body is in a second position with respect to said frame body, a second tension is provided to said at least one transmission cable via said tensioning body.

37. The surgical instrument assembly as set forth in claim 35, further comprising a lock body and a recess, wherein a final indexed position of said plurality of indexed positions constitutes a final use count of the surgical instrument assembly, and wherein, upon attainment of the final use count, said lock body is received in said recess and said input body is fully constrained from movement with respect to said frame body.

38. The surgical instrument assembly as set forth in claim 35, further comprising a cover body moveable with respect to said frame body, wherein, when said input body is in a first position with respect to said frame body, said cover body renders a flush port of the surgical instrument assembly inaccessible, and, when said input body is in a second position with respect to said frame body, movement of said cover body renders the flush port accessible.

39. A method of counting uses of a surgical instrument assembly, the method comprising:
moving an input body with respect to a frame body from a first position and to a second position, movement of said input body from the first position and to the second position prompting movement of an indexing body with respect to said frame body to an indexed position of a plurality of indexed positions, wherein, when said input body is in the first position, a use state of the surgical instrument assembly is established, and when said input body is in the second position, a reprocessing-ready state of the surgical instrument assembly is established; and
incrementing a use count of the surgical instrument assembly upon advancement of said indexing body to each of said plurality of indexed positions and upon the establishment of each reprocessing-ready states of the surgical instrument assembly.

40. A method of de-tensioning a surgical instrument assembly, the method comprising:
moving an input body to a first position with respect to a frame body, a use state of the surgical instrument assembly established when said input body is in the first position and a first tension provided to at least one transmission cable via a tensioning body; and
moving said input body to a second position with respect to said frame body, a non-use state of the surgical instrument assembly established when said input body is in the second position and a second tension provided to the at least one transmission cable via said tensioning body, the second tension being less than the first tension.

41. A method of de-tensioning a surgical instrument assembly as set forth in claim 40, wherein, when said input body is in the second position and the second tension is provided, a reprocessing-ready state of the surgical instrument assembly is established.

42. A handheld surgical instrument assembly, comprising:
a handle assembly, a frame body, a shaft body, a flush port in fluid communication with said shaft body, an end effector assembly, and at least one transmission cable extending from said handle assembly to said end effector assembly for transmitting actions to said end effector assembly;
an input body moveable with respect to said frame body;
an indexing body moveable with respect to said frame body, movement of said indexing body effecting a use count of the handheld surgical instrument assembly;
a tensioning body moveable with respect to said frame body, movement of said tensioning body effecting a tension of said at least one transmission cable; and
a cover body moveable with respect to said frame body, movement of said cover body effecting accessibility and inaccessibility of said flush port;
wherein, a single user input action to said input body and movement of said input body with respect to said frame body concurrently moves said indexing body with respect to said frame body, moves said tensioning body with respect to said frame body, and moves said cover body with respect to said frame body.

43. The handheld surgical instrument assembly as set forth in claim 42, wherein the single user input action and concomitant movements of said indexing body and of said tensioning body and of said cover body concurrently incites: i) advancement of the use count of the handheld surgical instrument assembly, ii) alteration of the tension of said at least one transmission cable, and iii) accessibility and inaccessibility of said flush port.

44. The handheld surgical instrument assembly as set forth in claim 42, wherein movements of said indexing body with respect to said frame body and of said tensioning body with respect to said frame body and of said cover body with respect to said frame body are concurrently prompted via movement of said input body.

45. The handheld surgical instrument assembly as set forth in claim 42, wherein, upon movement of said indexing body with respect to said frame body effecting a final use count of the handheld surgical instrument assembly, movement of said input body with respect to said frame body is fully constrained.

46. The handheld surgical instrument assembly as set forth in claim 42, further comprising a lock body and a recess, wherein, upon movement of said indexing body with respect to said frame body effecting a final use count of the handheld surgical instrument assembly, said lock body is received in said recess and movements of said indexing body and of said tensioning body and of said cover body with respect to said frame body is prevented.

47. The handheld surgical instrument assembly as set forth in claim 42, wherein movement of said tensioning body with respect to said frame body reduces the tension of the said at least one transmission cable and precludes the transmission of actions to said end effector assembly via said at least one transmission cable.

48. The handheld surgical instrument assembly as set forth in claim 42, wherein a use count indicia of the handheld surgical instrument assembly is displayed upon effecting the use count, said use count indicia representative of the use count of the handheld surgical instrument assembly.

49. The handheld surgical instrument assembly as set forth in claim 42, further comprising a retention body, said retention body preventing movement of said input body with respect to said frame body and, upon actuation of said retention body, said input body is moveable with respect to said frame body.

50. A surgical instrument assembly, comprising:
a frame body;
an input body moveable with respect to said frame body;
an indexing body prompted to move with respect to said frame body via said input body;
an obsolescence body moveable with respect to said frame body and, upon movement of said obsolescence body, movement of said input body with respect to said frame body is fully constrained, wherein movement of said obsolescence body and full constraint of said input body with respect to said frame body is prompted via attainment of a final indexed position of said indexing body or via an event trigger.
